(19) United States
(12) Patent Application Publication
(10) Pub. No.: US 2026/0207653 A1
(43) Pub. Date: Jul. 23, 2026

(54) O/W composition emulsified with OSA starch for use in glycogen storage disease

(71) Applicant: N.V. Nutricia, Zoetermeer (NL)

(72) Inventors: Leonardo Cornacchia, Utrecht (NL); Janna Willemina Woestenenk, Utrecht (NL); Bartholomeus Theodorus Gerardus Kompier, Utrecht (NL); Evan Abrahamse, Utrecht (NL); Simon David Anders, Liverpool (GB)

(73) Assignee: N.V. Nutricia, Zoetermeer (NL)

(21) Appl. No.: 19/568,995

(22) Filed: Mar. 17, 2026

Related U.S. Application Data
(63) Continuation of application No. PCT/EP2024/077127, filed on Sep. 26, 2024.

(30) Foreign Application Priority Data
Sep. 28, 2023 (EP) 23200459.8

(51) Int. Cl. A61K 31/718; A61K 9/107; A61K 47/42; A61P 3/08

(52) U.S. Cl. CPC A61K 31/718; A61K 9/107; A61K 47/42; A61P 3/08

(57) ABSTRACT

The invention relates to an emulsified O/W composition comprising emulsified lipid and OSA starch wherein the dry weight ratio of OSA starch to lipid is in the range from 1:3 to 1:5 and wherein less than 4 wt % protein is present based on total dry weight of the emulsified O/W composition and relates to the nutritional composition comprising the emulsified O/W composition and to the method of making these compositions and further relates to a nutritional composition for use in glycogen storage disease.

### FIELD OF THE INVENTION

The invention is in the field of medical food and relates to an emulsified O/W composition and a spray-dried composition comprising emulsified lipid and OSA starch and relates to the method of making these compositions and further relates to a nutritional composition for use in glycogen storage.

### BACKGROUND

Glycogen storage disease (GSD) is a rare inherited metabolic disorder where the human body is not able to properly break down glycogen to provide energy to the human body. Glycogen is one of the main sources for energy to the human body and is mainly stored in the liver. When the human body is in need of energy, enzymes in the liver break down glycogen to provide glucose. GSD patients are deficient or have mutations in one of the enzymes related to the break down stored glycogen. There are over 12 types of GSD classified based on the enzyme deficiency/mutations and the affected tissue. Symptoms of GSD involve hypoglycemia, enlarged liver, growth retardation, swollen belly, weak muscles, muscle cramping and pain and easy bruising.

GSD patients and idiopathic ketotic hypoglycemia (IKH) patients have to consume frequent carbohydrate rich meals along with uncooked corn starch in order to maintain normoglycemia during the 24 hours in a day. GSD cannot be prevented and there is no cure to GSD and thus treatment relates to disease management. In clinical practice, patients have to eat a carbohydrate-rich meal along with uncooked corn starch, e.g. maizena, every 2-4 hours to extend the fasting period (24/7). During nighttime some GSD patients are either on continuous nocturnal gastric drip feeding or have to eat 1-3 carbohydrate rich meals. This has an impact on the quality of life of the patient and family as it often results in sleep deprivation during the night.

The current nutritional solutions in the field are suboptimal, patients still have limited fasting periods affecting the quality of life. Furthermore, these nutritional compositions are incomplete, nutritionally poor, and lead to an imbalanced nutritional intake. Relatively high carbohydrate intake leads to excess glycogen storage in the liver and muscle. Excessive glucose peaks after food consumptions, with e.g. rapidly digestible carbohydrates, is unfavourable and may eventually lead to insulin resistance.

WO 2006/028122 describes the use of octenyl succinic anhydride-modified starch (OSA starch) for preventing and ameliorating obesity; and further describes use of octenyl succinic anhydride-modified starch as an inhibitor for blood glucose level elevation; or an agent for preventing and ameliorating diabetes. Here, OSA starch is used as dietary fibre to slow down digestion. The OSA starch is present in relatively high amounts in order to impart the desired inhibition on post-prandial blood glucose elevation which makes suchlike use of it in complete and balanced nutritional formulations for GSD patients challenging and less desired.

Wolf B W et al. “Effects of chemical modification on in vitro rate and extent of food starch digestion: an attempt to discover a slowly digested starch”, Journal Of Agricultural And Food Chemistry, 1999, vol. 47, 4178-4183 describe the application of slowly digestible starch (SDS) in type 2 diabetes and mentions the use of SDS, specifically uncooked/raw corn starch in GSD.

Gremse D A et al., “Efficacy of cornstarch therapy in type III glycogen-storage disease”, American Journal Of Clinical Nutrition, 1990, vol. 52, no. 4, 671-674 describes the use of cornstarch in GSD type III to prevent hypoglycemia/maintain normoglycemia and further mentions the use of protein supplementation in GSD patients. Yet, the challenge remains that GSD patients have to eat such a carbohydrate-rich meal, every 2-4 hours to extend the fasting period.

Food emulsions (e.g. in dietary supplements) are typically stabilised by proteins or low molecular weight emulsifiers like CITREM, lecithin etc. Such emulsions have good process and shelf-life stability but undergo significant destabilization during gastric digestion of the proteins due to the acid environment and the mechanical manipulation in the stomach and hence are suboptimal for GSD patients that benefit from prolonged gastric emptying by having a stable food emulsion.

EP0504055 describes such a liquid nutritional composition comprising a carbohydrate fraction with glucose polymers and slow-absorbing carbohydrates for use in GSD and further describes a process for making the composition by preparing an emulsion from a lipid phase to which two other fractions are added including protein and soluble fibres and the slow-absorbing carbohydrates which is represented as a model for present technology in the field of GSD management. However, protein is less stable in the gastric environment and thus protein-emulsified lipid is broken down earlier in the stomach resulting in an uneven dispersion of lipid, protein and carbohydrates in the food bolus and leading to a delayed cholecystokinin response which is unfavourable in GSD patients.

CN114847359 describes a process for obtaining a composition in which a core material solution is obtained comprising lipid, OSA starch in a ratio of 1:2.2, and a wall material solution is obtained comprising tapioca dextrin dissolved in water. It further describes the use of the composition in the dietary treatment of diabetes; the benefit of tapioca dextrin is that it would not lead to a rise in blood sugar level. However, high amounts of OSA starch may lead to digestive discomfort.

WO 2023/006894 describes the use of OSA starch to obtain an emulsified liquid composition wherein OSA starch, water and glucose syrup are mixed to obtain an aqueous phase to which the lipid is added. The emulsion is subsequently homogenized and spray-dried. WO 2023/006894 further mentions the addition of protein to the pre-emulsion which can result in an uneven dispersion of lipid, protein and carbohydrates in the food bolus.

Despite the effort of research focused on the intrinsic properties of uncooked corn starch on glucose metabolism, there is a lack of information on the effect of the food matrix in a composition on the glucose release. There remains a need in the field to develop a nutritional composition including a slowly digestible starch source with slow glucose release characteristics that particularly is resistant to gastric conditions present in the stomach.

### SUMMARY OF THE INVENTION

For patients at risk for hypoglycaemia, such as GSD patients, controlled gastric emptying resulting in a constant release of nutrients overtime and slow carbohydrate digestion are required for reducing and/or treating the disease, i.e. by means of disease management. Proper disease management through a diet rich in slowly digestible carbohydrates ensures that the GSD patient remains at normoglycemia for a longer period. Additionally, it is important that the lipid consumed in diets for GSD patients remains sufficiently emulsified under gastric conditions in order to be administered in the duodenum at a steady rate. The inventors developed a gastric-stable oil-in-water (O/W) emulsion and composition using OSA starch as emulsifier, whereof the glucose release is slower which benefits such patients. More specifically, the inventors have developed a food emulsion which can remain dispersed in a liquid at in vitro gastric conditions by using OSA starch as an emulsifier. Such gastric-stable O/W compositions are important to influence the rate of stomach emptying and control the overall nutrient digestion in a food product or supplement.

It is particularly preferred to also keep the amounts of digestible carbohydrates during emulsification at reduced or limited levels, preferably less than 10 wt %, more preferably less than 4 wt % digestible carbohydrates, based on the total dry weight of the composition; alternatively worded, the emulsified O/W composition preferably comprises less than 10 wt % digestible carbohydrates, more preferably less than 4 wt %, based on the total dry weight of the emulsified O/W composition. The advantage of using limited amounts of digestible carbohydrates during emulsification is to prevent interference of the digestible carbohydrates in the coating of OSA starch on the lipid globules which can lead to instability issues as upon administration, as the digestible carbohydrates are more easily digested and can thus lead to unequal dispersion of the composition in the stomach.

In the present invention, the inventors have developed an emulsified O/W composition and a spray-dried composition characterized by a matrix comprising a gastric-stable emulsion using OSA starch as emulsifier and an encapsulating agent which encapsulates the gastric-stable emulsion which can be used in nutritional compositions for e.g. GSD patients.

The inventor's findings aim to provide a prolonged and substantially constant glucose response for patients, to remain at normoglycemia for a longer period. Thereto, the present invention provides a method for preparing an emulsified oil-in-water (O/W) composition comprising emulsifying lipid with a first aqueous phase and octenyl succinic anhydride substituted starch (OSA) starch to obtain an emulsified O/W composition, wherein the dry weight ratio of OSA starch to lipid is in the range from 1:3 to 1:5 and wherein less than 4 wt % protein is present, based on total dry weight of the emulsified O/W composition.

According to the inventors, it is important that in the method the presence of protein is limited during the emulsification of lipid with OSA starch to prevent interference with migration of OSA starch to the O/W emulsion interface and/or positioning of protein on the O/W emulsion interface. The inventors have surprisingly found that presence of protein or other surface-active components at the emulsion interface are less resilient to the gastric environment and can destabilize the emulsion during gastric digestion. Hence, emulsification of lipid with OSA starch without the presence of protein at the O/W interface allows to obtain a more stable emulsion and an improved coating of OSA starch around lipid globules. In this manner, the nutritional composition upon reconstitution comprising the O/W emulsion remains stable during gastric digestion. Hence, it is important that protein presence is limited at the O/W interface in the emulsified O/W composition, therefore the lipid is emulsified with OSA starch, while proteins are added after the emulsion is formed e.g., before pasteurization and spray-drying or either added via dry-blending after spray-drying.

In view of the above, the present invention also provides an emulsified O/W composition obtainable by the method according to the invention, wherein the O/W composition comprises a lipid phase comprising lipid globules coated with OSA starch. The coating prevents the lipid globules from making contact with an outer comprising protein, and also prevents the lipid globules from making contact with protein that can be adsorbed onto or forms a separate layer onto the OSA starch coating.

The present invention further provides a nutritional spray dried composition comprising lipid, OSA starch, an encapsulating agent and optionally protein, wherein the composition comprises lipid-containing particles which have a lipid core and wherein said lipid core is coated with OSA starch, and wherein the lipid core and OSA starch coating comprise less than 4 wt % protein altogether, based on total dry weight in the composition.

The coating prevents the lipid core from making contact with an outer layer comprising protein, and also prevents the lipid core from making contact with protein that can be adsorbed onto or forms a separate layer onto the OSA starch coating. Furthermore, the inventors have found that such a gastric stable O/W composition in a specific matrix design comprising resistant maltodextrin as encapsulating agent for spray-drying further benefits GSD patients in further controlling the rate of carbohydrate release because it modulates digestion of the carbohydrates in favour of prolonged normoglycemia. In this regard, the lipid-containing particle, comprising the OSA starch coating, is provided with a further coating by the encapsulating agent. The nutritional composition of the present invention reduces the rate of carbohydrate glucose release via reduced stomach emptying and reduced enzymatic starch hydrolysis rates, which provides a prolonged glucose response that benefits GSD patients to maintain normoglycemia for a longer period of time. In this regard, the digestible carbohydrates are preferably dry-blended with the nutritional spray-dried composition.

Associated with the prolonged or sustained glucose response, the present invention further provides the nutritional composition as described here above for use in reducing and/or treating carbohydrate-related metabolic disorders, and which disorders are associated with an impaired glucose response. Worded differently, the invention also relates to the use of the nutritional composition as described herein in the manufacture of a product for reducing and/or treating carbohydrate-related metabolic disorders. Related therewith, the invention also pertains to a method for reducing and/or treating carbohydrate-related metabolic disorders in a subject in need thereof, the method comprising administering the nutritional composition as described herein. In the above, the method or use is preferably for use in reducing and/or treating diabetes, glycogen storage disease (GSD), fatty acid oxidation disorders (FAOD) and/or idiopathic ketotic hypoglycemia (IKH), more preferably for use in liver related GSD subtype 0, III, VI and IX.

To this end, the slow release of glucose is modulated in favour of prolonged normoglycemia by the components in the matrix design:
1. An OSA-starch stabilized emulsion is encapsulated by a resistant maltodextrin shell. The OSA starch oil-in-water (O/V) emulsion remains dispersed under gastric conditions and thereby ensures a homogenous distribution of the lipid throughout the gastric digesta. Therewith, lipid and other nutrients will be emptied from the stomach together. At arrival in the small intestine the lipid digestion products elicit a hormonal feedback response (CCK) controlling gastric emptying. The resistant maltodextrin avoids the rapid carbohydrate digestion of the conventional food encapsulants (e.g. conventional maltodextrins used in spray drying) and therewith the overall glucose release from the composition is limited.
2. A digestible carbohydrate source, preferably a starch, comprising a high fraction of slowly digestible starch (SDS) is used in the nutritional composition as primary source of glucose in the composition. The preservation of the starch structure allows to achieve a slower carbohydrate digestion, which together with a gastric-stable emulsion ensures slow and more sustained release of glucose during digestion.

In this regard, the transit from the stomach to duodenum is regulated, amongst others, by the amount of calories and by biochemical events triggered by free fatty acids. A gastric-unstable composition rapidly coalesces in the gastric environment and forms a lipid layer on the top of an aqueous phase comprising carbohydrates. Consequently, lipid is digested later and enters the duodenum after the aqueous phase. The aqueous phase with carbohydrates is lower in calories (±4 kcal per gram) than the lipid layer (9 kcal per gram lipid) and therefore the gastric emptying is faster. Also, the coalescence of oil droplets reduces the surface area for lipolysis, resulting in a decreased free fatty acid release.

In vivo gastrointestinal digestion of a gastric-stable composition may lead to a decreased gastric emptying rate of the gastric-stable composition, which may be attributed to the higher stimulation of CCK release into the blood plasma. The inventors have found OSA starch to be a suitable emulsifier in the formulation of a gastric-stable composition, whereby the oiling-off (demulsification) is reduced and a stable droplet size distribution is maintained with fewer coalescence throughout in vitro gastric digestion experiments. Furthermore, the inventors have found that resistant maltodextrin as encapsulating agent does not impact blood glucose levels upon digestion and thus in combination with OSA starch in a nutritional composition benefits slow glucose release and maintaining normoglycemia.

Lipid and lipid digestion products preferably are released in the duodenum throughout the gastric transit time to promote the CCK pathway within 3-5 hours after food consumption. Fasting motility returns 3-5 hrs after registration of a normal sized meal, expelling particles of any size left in the stomach, The inventors have found that the emulsified O/W composition according to the invention and the nutritional composition according to the invention are able to stay stable for at least 3 hours in the gastric environment under gastric conditions.

In this regard, a gastric-unstable O/W composition rapidly coalesces in a gastric environment and forms an oil layer on the top of an aqueous phase. Thus, the emulsified O/W composition obtainable by the method according to the invention is preferably gastric-stable and refers to the stability of the composition under gastric conditions. The gastric conditions include an increasing acidic pH over time in simulated stomach conditions in the presence of gastric enzymes for 120 minutes as according to the standardised in vitro digestion method described in Mulet-Cabero et al., A standardised semi-dynamic in vitro digestion method suitable for food—an international consensus, Food Funct., 2020, 11, 1702-1720. In a simplified embodiment, gastric conditions refer to an acidic pH between 2.5-4 for 120 minutes at 37° C. in the presence of gastric enzymes pepsin at a concentration of 4000 U mL-1 and gastric lipase at a concentration of 120 U mL-1.

The emulsified O/W composition is an emulsified composition which does not show a phase separation or oiling-off (demulsification) after 4 hours at 37° C. compared to a composition which is emulsified with whey protein isolate. In addition, the emulsified O/W composition has a stable droplet size distribution when exposed to simulated in vitro human gastric conditions as described by Mulet-Cabero et al. (2020) with fewer coalescence and thereby lipid droplets remain dispersed in the in the stomach compared to an emulsified O/W composition which is emulsified with hydrolysed whey protein.

Without being bound to any theories, it is hypothesized that free fatty acids comprised in the lipid core in the emulsified nutritional composition upon digestion bind to G-coupled receptors (GPR) located in the enteroendocrine cells (EEC) in the small intestinal epithelia thereby affecting satiety and gastrointestinal motor activity by the release of the peptides, such as cholecystokinin (CCK), glucagon-like peptide 1 (GLP-1) and peptide tyrosine tyrosine (peptide YY). The increased exposure of these peptides may result in a decreased gastric emptying rate whereby a controlled gastric emptying rate, and retention of nutritional food in the stomach ensures a gradual digestion in the gastro-intestinal tract.

It is important that the lipid-comprising emulsion remains dispersed at gastric conditions to influence the rate of stomach emptying and control the overall nutrient digestion in GSD patients. The stability is considered to be attributed to the steric repulsion which is generated by the branched structure of OSA-starch. In addition to this, it is hypothesized that the presence of OSA starch in a nutritional product poses a great hindrance to the gastric enzymes thereby indicating that the lipid globules coated with the OSA starch can resist the attack of the ionic strength and the a-amylase in the simulated salivary fluid. It is hypothesized that the stability against gastric conditions is derived from the presence of carboxyl groups in OSA-starch providing high total net negative charge in emulsions and contributing to a more rigid and compact surface resisting the coalescence of the globules.

This improved gastric stability using OSA starch as emulsifier ensures a homogenous distribution of the lipid throughout the gastric digesta. Therewith, lipid and other nutrients will be emptied from the stomach together. At arrival in the small intestine the lipid digestion products elicit a hormonal feedback response (CCK) slowing down gastric emptying of the stomach and therewith manages the overall glucose release. An unstable emulsion, e.g. using protein as emulsifier, the protein covering the lipid globules is already partially digested or broken down in the stomach due to the acidic environment and thereby releasing lipid droplets. As a consequence, lipid layers on top of the chyme, after the aqueous phase with the other nutrients would already be emptied and the feedback response to control gastric emptying would thereby be delayed. Furthermore, the reduced surface area of an unstable emulsion would limit lipolysis, and release of the lipid digestion products that elicit the hormonal feedback response. This is all avoided with the use of OSA starch according to the invention in the O/W composition and in the nutritional composition. As fasting motility returns 3-5 hrs after registration of a normal sized meal, expelling particles of any size left in the stomach, the gastric-stable composition ideally stays stable for at least 3 hours after ingestion.

Furthermore, it is important that the encapsulating agent does not impact blood glucose levels or increase glucose digestion in the stomach and is thus different from regular maltodextrin, which is rapidly broken down into glucose and absorbed in the small intestine. Resistant maltodextrin passes through the small intestine without being fully digested. Instead, it is fermented by the gut microbiota in the large intestine, leading to the production of short-chain fatty acids (SCFAs) and other beneficial metabolites. By producing a slow release formulation, the carbohydrate digestion rate is controlled and therewith extends the fasting time after ingestion of the nutritional composition, thus improving the quality of life for patients in need thereof, such as GSD patients.

### LIST OF FIGURES

The present invention will be discussed in more detail below, with reference to the attached figures.

FIG. 1. The particle size distribution of reconstituted base powder with pH 7 (1A) and acidic reconstituted base powder with pH 4 (1B) at t=0: directly after reconstitution and t=4 h: 4 hours after reconstitution.

FIG. 2. The particle size distribution after reconstitution of the spray-dried composition comprising the OSA starch-stabilized emulsion and the spray-dried composition comprising the WPI-stabilized emulsion.

FIG. 3. The particle size distribution after reconstitution of the spray-dried composition comprising the OSA starch-stabilized emulsion (3A) and the spray-dried composition comprising the WPI-stabilized emulsion (3B) during in vitro simulated oral and gastric digestion for 2 hours.

FIG. 4. Glucose (4A) and paracetamol (4B) profiles of the three different treatment groups (UCCS, neutral prototype and acidic prototype) wherein blood samples were collected and glucose and paracetamol levels were measured at 30-minute intervals for 6 hours post-prandial.

FIG. 5. Particle size distribution of the reconstituted OSA starch-stabilized emulsion—base powder A and WPI stabilized emulsion—Base powder B.

FIG. 6. Particle size distribution of base powder A during 120 min gastric digestion. Samples were collected after 24, 48, 72 and 120 min.

FIG. 7. Particle size distribution of base powder B during gastric digestion, samples were collected after 24, 48, 72 and 120 min.

The particle size distributions depicted in the Figures are volume-weighted distributions determined with laser diffraction using a laser-light diffraction unit (Mastersizer 2000, Malvern Instruments Ltd, Worcestershire, UK) for example by the method described in Michalski et al., 2001, Lait, 81, 787-789. The size distribution was obtained using polydisperse analysis and particle size measurements were recorded as average mean diameter D50 and volume mean diameter (D4,3) to observe the effect of gastric digestion on the change of the oil droplet size distribution as further explained in the examples.

### LIST OF PREFERRED EMBODIMENTS

1. A method for preparing an emulsified oil-in-water (O/W) composition comprising emulsifying lipid with a first aqueous phase and octenyl succinic anhydride substituted starch (OSA) starch to obtain an emulsified O/W composition, wherein the dry weight ratio of OSA starch to lipid is in the range from 1:3 to 1:5 and wherein less than 4 wt % protein is present, based on total dry weight of the emulsified O/W composition.
2. The method according to embodiment 1, wherein the dry weight ratio of OSA starch to lipid is in the range from 1:3.5 to 1:4.5.
3. The method according to embodiment 1 or 2, wherein emulsification of lipid with the first aqueous phase and OSA starch is performed at a pH in the range of 3-5.5, preferably in the range of 3.5 to 5.
4. An emulsified O/W composition obtainable by any one of embodiments 1-3, wherein the O/W composition comprises a lipid phase comprising lipid globules coated with OSA starch.
5. The method according to any one of embodiments 1-3, wherein the method further comprises:
   adding an encapsulating agent to the emulsified O/W composition;
   optionally adding a second aqueous phase comprising protein to the emulsified O/W composition and optionally pasteurizing the emulsified O/W composition;
   spray-drying the emulsified O/W composition, to obtain a spray-dried composition;
wherein the spray-dried composition comprises lipid, OSA starch, the encapsulating agent and optionally protein, wherein the spray-dried composition comprises lipid-containing particles which have a lipid core and wherein said lipid core is coated with OSA starch, and wherein the lipid core and OSA starch coating comprise less than 4 wt % protein altogether, based on total dry weight of the spray-dried composition.
6. The method according to embodiment 5, wherein the encapsulating agent is added to the first aqueous phase, and/or to the emulsified O/W composition and/or to the second aqueous phase and/or wherein the encapsulating agent comprises resistant maltodextrin, preferably the encapsulating agent is resistant maltodextrin.
7. The method according to embodiment 5 or 6, wherein less than 4 wt % digestible carbohydrates based on total dry weight is present in the composition prior to being subjected to spray-drying.
8. A nutritional composition comprising lipid, OSA starch, an encapsulating agent and optionally protein, wherein the composition comprises lipid-containing particles which have a lipid core and wherein said lipid core is coated with OSA starch, and wherein the lipid core and OSA starch coating comprise less than 4 wt % protein altogether, based on total dry weight of the composition.
9. The nutritional composition according to embodiment 8, wherein the composition is a powder.
10. The nutritional composition according to embodiment 8 or 9, wherein the composition comprises less than 10 wt %, preferably less than 8 wt %, more preferably less than 4 wt % lipid-containing particles of which the core is not coated with OSA starch based on total lipid weight in the nutritional composition.
11. The nutritional composition according to any one of embodiments 8-10, wherein the composition comprises based on total dry weight of the composition:
   at least 10 wt % lipid; preferably the lipid comprises sunflower oil;
   2-6 wt % OSA starch;
   2-15 wt % encapsulating agent, preferably the encapsulating agent comprises resistant maltodextrin;
and wherein the composition further comprises:
   25-75 wt % digestible carbohydrates wherein the carbohydrates comprises at least 90 wt % native maize starch based on total dry weight of the carbohydrates.
12. The nutritional composition according to embodiment 11, wherein the composition comprises protein, preferably wherein the composition comprises 10-35 wt % protein based on total dry weight of the nutritional composition.
13. The nutritional composition according to embodiment 11 or 12, wherein the native maize starch comprises at least 55 wt % slowly digestible starch (SDS) and less than 20 wt % resistant starch (RS) based on total dry weight of the native maize starch.
14. The nutritional composition according to any one of embodiments 11-13, for use in reducing and/or treating carbohydrate-related metabolic disorders, preferably for use in reducing and/or treating diabetes, glycogen storage disease (GSD), fatty acid oxidation disorder (FAOD) and/or idiopathic ketotic hypoglycemia (IKH), more preferably for use in liver related GSD subtype 0, III, VI and IX.
15. The nutritional composition for use according to embodiment 14, wherein reducing and/or treating diabetes, GSD, FAOD, and/or IKH comprises preventing and/or reducing one or more of insomnia or sleep deprivation, impaired normoglycemia, hypoglycemia during fasting periods, hyperinsulinism, insulin resistance, gastrointestinal side effects comprising one or more of bloating, gas, diarrhoea and wherein reducing and/or treating diabetes, GSD, FAOD, and/or IKH further comprises one or more of maintaining normoglycemia, prolonging gastric emptying rate, prolonging carbohydrate digestion, prolonging glycolytic breakdown during digestion.

### DETAILED DESCRIPTION

### List of Abbreviations

GSD=liver related glycogen storage disease. The focus is on patients with a ketotic subtype of GSD (subtype 0, III, VI and IX))
IKH=idiopathic ketotic hypoglycemia
LCFA=long chain fatty acids
SFO=sunflower oil
OSA starch=octenyl succinic anhydride starch
SDS=slowly digestible starch
RDS=rapidly digestible starch
RS=resistant starch

### List of Definitions

Throughout this application, the following terminology and abbreviations may be used. “Nutritional composition” means a substance or formulation that satisfies at least a portion of a subject's nutrient requirements. The terms “nutritional(s)”, “nutritional formula(s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, concentrates, suspensions, or ready-to-use forms of enteral formulas, oral formulas, formulas for infants, formulas for paediatric subjects, formulas for children, growing-up milks and/or formulas for adults.

The term “nutritionally complete” or “nutritionally complete composition” refer to a composition comprising the essential nutrients including essential amino acids, essential fatty acids, vitamins, and minerals, or adequate calories to meet the energy needs of the individual, based on their age, sex, activity level, and other factors, or balanced macronutrient in terms of carbohydrates, proteins, and lipids—to support various bodily functions.

The terms “treatment”, “treat”, “to alleviate”, “reduce” include therapeutic or disease-modifying treatment, including therapeutic measures that slow down, lessen symptoms of, and/or halt progression of the diagnosed pathologic condition or disorder as defined herein, preferably to reduce (the occurrence of) symptoms, and/or improve quality of life by improved daily living, and/or improve sleeping pattern in subjects suffering from the disease(s) as defined herein; The term ‘treatment’ includes treatment of patients at risk of contracting the disease or suspected to have contracted the disease, as well as patients who are ill or have been diagnosed as suffering from the disease or the medical condition as defined herein. The term does not imply that a subject is treated until total recovery.

The terms “treatment,” “treat” and “to alleviate” are also intended to include the potentiation or otherwise enhancement of primary therapeutic measure(s).

The terms “lipid”, “lipid fraction”, “lipid component”, “lipid ingredient” are synonyms and used interchangeably.

The terms “stable emulsion”, “stabilized emulsion”, “OSA starch-stabilized emulsion” are used interchangeably and refer to an emulsion stabilized with OSA starch according to the invention.

The term “gastric-stable” refers to the stability under human gastric conditions of the emulsion stabilized with OSA starch according to the invention. Herein, the gastric conditions refer to an increasingly acidic pH over time in simulated human stomach conditions in the presence of gastric enzymes for 120 minutes. as according to the standardised in vitro digestion method described in Mulet-Cabero et al., A standardised semi-dynamic in vitro digestion method suitable for food—an international consensus, Food Funct., 2020, 11, 1702-1720.

The term “oil-in-water composition” or “O/W composition” refer to a composition of lipid in water and/or in an aqueous solution wherein the composition comprises a lipid phase (i.e. oil phase) and a water phase.

The term “emulsified O/W composition”, refers to an emulsified composition comprising the “gastric-stable emulsion”, i.e. the emulsified O/W composition comprises the OSA starch-stabilized emulsion or in other words comprises the emulsion stabilized with OSA starch according to the invention. The emulsified O/W composition comprises a lipid phase comprising lipid globules coated with OSA starch. The coating prevents the lipid globules from making contact with an outer layer comprising protein, and also prevents the lipid globules in the O/W composition from making contact with protein that can be adsorbed onto or forms a separate layer onto the OSA starch coating. The emulsified O/W composition as used herein is not a nutritionally complete composition, i.e. the emulsified O/W composition does not contain the essential nutrients including essential amino acids, essential fatty acids, vitamins, and minerals, or adequate calories to meet the energy needs of the individual, based on their age, sex, activity level, and other factors, or balanced macronutrient in terms of carbohydrates, proteins, and fats—to support various bodily functions.

The term “(nutritional) spray-dried composition” or “powdered composition” as used herein refers to a nutritional powdered composition which has been spray-dried, and can be dry blended or mixed with additional components to obtain the final nutritional composition. The spray-dried composition as used herein refers to a composition that has been subjected to spray-drying to obtain a powder, i.e. a powdered composition.

The term “powder” as used herein refers to fine, individual particles that are preferably smaller than 1 mm in diameter having a higher flowability and solubility compared to granules which have a coarser structure and a particle size typically ranging between 1 to 10 mm based on volume.

In the context of the invention, the term “essentially free” refers to the absence of the mentioned ingredient or if present, less than 4 wt %, more preferably less than 3 wt %, even more preferably less than 1 wt % of the mentioned ingredient, based on total (dry) weight of the composition, most preferably below detectable levels. In particular, the term “essentially free from protein” refers to the absence of protein or the presence of less than 4 wt % protein, preferably less than 3 wt %, more preferably less than 1 wt % based on total dry weight of the emulsified O/W composition. Or in other words, wherein less than 4 wt %, preferably less than 3 wt %, more preferably less than 1 wt % protein based on total dry weight of the nutritional composition is present in the first aqueous phase and in the emulsified O/W composition together. Regarding the emulsified O/W composition specifically, less than 4 wt %, preferably less than 3 wt %, more preferably less than 1 wt % based on total dry weight is present in lipid phase comprising lipid globules coated with OSA starch. Regarding the spray-dried composition and the final nutritional composition, “essentially free from protein” refers to less than 4 wt % protein, preferably less than 3 wt %, more preferably less than 1 wt % protein is present in the lipid-containing particle comprising a lipid-containing core and OSA starch coating surrounding the lipid core based on total dry weight of the nutritional composition. Or more specifically, when the nutritional composition comprises protein, wherein less than 4 wt % protein, preferably less than 3 wt %, more preferably less than 1 wt % protein is present in the lipid-containing particle comprising a lipid-containing core and OSA starch coating surrounding the lipid core based on total protein weight in the nutritional composition.

Also, in context of the invention, the terms “coated with OSA starch”, “coating of OSA starch”, “the coating with OSA starch” and the like refer to the OSA starch being on the surface of the lipid core/lipid globule. Preferably, the terms refer to the lipid core or lipid globule coated with OSA starch, which OSA starch coating prevents the lipid core or lipid globule from making contact with an outer layer comprising protein and/or prevents the lipid core or lipid globule from making contact with protein that is adsorbed onto or forms a separate layer onto the OSA starch coating. In a further preferred embodiment, the OSA starch coating is a layer of OSA starch surrounding the lipid core/lipid globule.

The term “encapsulating agent” or “spray-drying aiding agent” or “spray-drying aiding carbohydrate” or also called the “spray-drying agent”, refers to an ingredient in the composition used for coating a liquid composition in a spray-drying step, specifically the emulsified O/W composition that is subjected to spray-drying, to obtain the spray-dried composition as defined herein. In more detail, it refers to the encapsulating agent coating the OSA starch-stabilized emulsion present in the nutritional composition.

Furthermore, the terms “coated with the encapsulating agent”, “coating of the encapsulating agent”, “the coating with the encapsulating agent” and the like refer to the encapsulating agent being on the surface of the lipid-containing particles and optionally, on the outer layer comprising protein and optionally other further ingredients, which outer layer surrounds the lipid-containing particles. In this regard, the coating with the encapsulating agent forms an additional coating on the lipid-containing particle which latter comprises an OSA starch coating. Preferably, the terms refer to the lipid-containing particles and/or outer layer surrounding the lipid-containing particles coated with the encapsulating agent which encapsulating agent-coating prevents the lipid-containing particles and/or the outer layer from making contact with a further ingredient that is adsorbed onto or forms a separate layer onto the encapsulation agent. In a further preferred embodiment, the encapsulating agent-coating is a layer of the encapsulating agent surrounding the lipid-containing particles. More preferably, the encapsulating agent covers at least 75%, more preferably at least 85%, most preferably at least 90% of the surface of the lipid-containing particles and/or optionally of the surface of the outer layer comprising protein and optionally other further ingredients.

The term “food-grade” as used herein, refers to the product, material or additive as being safe for consumption, meaning it can be used for consumption without posing any health risks. The substances that are added to food products during processing or production are carefully regulated and approved for use in food by regulatory authorities, such as the Food and Drug Administration (FDA) in the United States or the European Food Safety Authority (EFSA) in the European Union.

All percentages are by weight unless otherwise stated.

The term “dry weight” as used herein, refers to being water-free, or in other words to the weight not including water.

In this document and in its claims, the verb “to comprise” and its conjugations is used in its non-limiting sense to mean that items following the word are included, but items not specifically mentioned are not excluded. Further when used herein the term weight percent (wt %) is the percentage of weight based on the total dry weight. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

Any reference to prior art documents in this specification is not to be considered an admission that such prior art is widely known or forms part of the common general knowledge in the field.

The invention will now be described in further details.

The present invention first and foremost relates to the method for preparing an oil-in-water (O/W) composition wherein lipid is emulsified with an aqueous phase and OSA starch. In further aspect, the invention concerns an O/W composition obtainable by said method and concerns the nutritional composition comprising said O/W composition. In a further aspect, the invention concerns the medical use of said nutritional composition. Before defining each of the aspects of the invention in more detail, the various features of the invention are defined in more detail. It is noted that the various aspects, features, examples and embodiments described in the present application may be compatible and/or combined together.

In a preferred aspect, the present invention relates to a method for preparing an emulsified oil-in-water (O/W) composition comprising emulsifying lipid with a first aqueous phase and octenyl succinic anhydride substituted starch (OSA) starch to obtain an emulsified O/W composition, wherein the dry weight ratio of OSA starch to lipid is in the range from 1:3 to 1:5 and wherein less than 4 wt % protein is present, based on total dry weight of the emulsified O/W composition. It is particularly preferred to also keep the amounts of digestible carbohydrates during emulsification at reduced or limited levels, preferably less than 10 wt %, more preferably less than 4 wt % digestible carbohydrates, based on the total dry weight of the O/W composition; alternatively worded, the emulsified O/W composition preferably comprises less than 10 wt % digestible carbohydrates, more preferably less than 4 wt %, based on the total dry weight of the emulsified O/W composition. Preferably, method further comprises:
adding an encapsulating agent to the emulsified O/W composition;
optionally adding a second aqueous phase comprising protein to the emulsified O/W composition and optionally pasteurizing the emulsified O/W composition;
spray-drying the emulsified O/W composition, to obtain a spray-dried composition;
   wherein the spray-dried composition comprises lipid, OSA starch, the encapsulating agent and optionally protein, wherein the spray-dried composition comprises lipid-containing particles which have a lipid core and wherein said lipid core is coated with OSA starch, and wherein the lipid core and OSA starch coating comprise less than 4 wt % protein altogether, based on total dry weight in the spray-dried composition. Preferably, wherein the OSA starch coating is provided with a further coating by the encapsulating agent. Preferably, the encapsulating agent has a glycaemic index in the range of 0 to 50, preferably in the range of 0 to 25, more preferably the encapsulating agent comprises, more preferably is, resistant maltodextrin. In a further preferred embodiment, less than 10 wt %, more preferably less than 4 wt % digestible carbohydrates based on total dry weight is present in the emulsified O/W composition prior to being subjected to spray-drying.

In a further preferred aspect, the invention concerns an emulsified O/W composition obtainable by the method for preparing an emulsified O/W composition, wherein the O/W composition comprises a lipid phase comprising lipid globules coated with OSA starch. In a preferred embodiment, dry weight ratio of OSA starch to lipid in the emulsified O/W composition is in the range from 1:3.5 to 1:4.5. Preferably, wherein the emulsified O/W composition comprises less than 10 wt %, more preferably less than 4 wt % digestible carbohydrates based on total dry weight of the emulsified O/W composition.

In a further preferred aspect, the invention concerns a nutritional spray-dried composition comprising lipid, OSA starch, an encapsulating agent and optionally protein, wherein the composition comprises lipid-containing particles which have a lipid core and wherein said lipid core is coated with OSA starch, and wherein the lipid core and OSA starch coating comprise less than 4 wt % protein altogether, based on total dry weight of the composition. In a preferred embodiment, the encapsulating agent forms an additional coating on the lipid-containing particles. In an alternative embodiment, when the nutritional spray-dried composition comprises protein, preferably less than 4 wt % protein, preferably less than 3 wt %, more preferably less than 1 wt % protein is present in the lipid-containing particle comprising a lipid-containing core and OSA starch coating surrounding the lipid core based on total protein weight in the nutritional composition. Preferably, wherein the coating of OSA starch prevents the lipid core from making contact with an outer layer comprising protein and/or prevents the lipid core from making contact with protein that is adsorbed onto or forms a separate layer onto the OSA starch coating. In a further preferred aspect, the lipid-containing particles, comprising the OSA starch coating, are coated with the encapsulating agent, more preferably, wherein the encapsulating agent prevents the lipid-containing particles from making contact with a further ingredient that is adsorbed onto or forms a separate layer onto the encapsulation agent.

Preferably, the final nutritional composition comprises lipid, digestible carbohydrates, OSA starch, an encapsulating agent and optionally protein, wherein the composition comprises based on total dry weight of the composition:
at least 10 wt % lipid; preferably the lipid comprises sunflower oil;
1-14 wt %, preferably 2-10 wt %, more preferably 2-6 wt % OSA starch;
25-75 wt % digestible carbohydrates preferably wherein the digestible carbohydrates comprises at least 55 wt % slowly digestible starchbased on total dry weight of the digestible carbohydrates;
2-15 wt % encapsulating agent, preferably the encapsulating agent comprises resistant maltodextrin.

In a further preferred aspect, the invention concerns a nutritional spray-dried composition comprising lipid, OSA starch, an encapsulating agent and optionally protein, wherein the composition comprises lipid-containing particles which have a lipid core and wherein said lipid core is coated with OSA starch, and wherein the lipid core and OSA starch coating comprise less than 4 wt % protein altogether, based on total dry weight in the composition for use in reducing and/or treating carbohydrate metabolic disorders, preferably for use in reducing and/or treating diabetes, glycogen storage disease (GSD), fatty acid oxidation disorder (FAOD) and/or idiopathic ketotic hypoglycemia (IKH), more preferably for use in liver related GSD subtype 0, III, VI and IX.

The invention can also be worded as a method for reducing and/or treating carbohydrate metabolic disorders, preferably reducing and/or treating diabetes, glycogen storage disease (GSD), fatty acid oxidation disorder (FAOD) and/or idiopathic ketotic hypoglycemia (IKH), more preferably liver related GSD subtype 0, III, VI and IX; said method comprising administering to the patient in need thereof a nutritional spray-dried composition comprising lipid, OSA starch, an encapsulating agent and optionally protein, wherein the composition comprises lipid-containing particles which have a lipid core and wherein said lipid core is coated with OSA starch, and wherein the lipid core and OSA starch coating comprise less than 4 wt % protein altogether, based on total dry weight in the composition.

The invention can also be worded as the use of OSA starch for the manufacture of a nutritional spray-dried composition for use in reducing and/or treating carbohydrate metabolic disorders, preferably reducing and/or treating diabetes, glycogen storage disease (GSD), fatty acid oxidation disorder (FAOD) and/or idiopathic ketotic hypoglycemia (IKH), more preferably liver related GSD subtype 0, III, VI and IX, wherein the nutritional composition comprises lipid, OSA starch, an encapsulating agent and optionally protein, wherein the composition comprises lipid-containing particles which have a lipid core and wherein said lipid core is coated with OSA starch, and wherein the lipid core and OSA starch coating comprise less than 4 wt % protein altogether, based on total dry weight in the composition.

### Method for Preparing the O/W Composition and the Spray-Dried Composition According to the Invention

An embodiment of the invention includes the method for preparing an oil-in-water (O/W) composition wherein lipid is emulsified with a first aqueous phase and OSA starch to obtain the emulsified O/W composition and wherein emulsification of lipid with a first aqueous phase and OSA starch is performed essentially free from protein. In a preferred embodiment, less than 4 wt % protein is present based on total dry weight of the emulsified O/W composition, more preferably less than 3 wt %, most preferably less than 1 wt % based on total dry weight of the O/W composition. In addition, preferably the amounts of digestible carbohydrates during emulsification are at reduced or limited levels, preferably less than 10 wt %, more preferably less than 4 wt % digestible carbohydrates, based on the total dry weight of the O/W composition; alternatively worded, the emulsified O/W composition preferably comprises less than 10 wt % digestible carbohydrates, more preferably less than 4 wt %, based on the total dry weight of the emulsified O/W composition.

In a further embodiment, the method for preparing the O/W composition as detailed here above further comprises the steps of:
adding an encapsulating agent to the emulsified O/W composition;
optionally adding a second aqueous phase comprising protein to the emulsified O/W composition and optionally pasteurizing the emulsified O/W composition;
spray-drying the emulsified O/W composition, to obtain a spray-dried composition;
   wherein the spray-dried composition comprises lipid, OSA starch, the encapsulating agent and optionally protein, wherein the spray-dried composition comprises lipid-containing particles which have a lipid core and wherein said lipid core is coated with OSA starch, and wherein the lipid core and OSA starch coating comprise less than 4 wt % protein altogether, based on total dry weight in the spray-dried composition. Preferably, wherein the encapsulating agent forms an additional coating on the lipid-containing particles.

In typical production of spray-dried compositions, the water-soluble/hydrophilic components being protein, an emulsifier, and optionally carbohydrates are dispersed and homogenised with lipid to form an emulsified aqueous phase, which is then pasteurized and spray-dried. However, in the method according to the invention, the emulsification is performed essentially free from protein, preferably less than 4 wt %, more preferably less than 3 wt %, most preferably less than 1 wt % protein is present based on total dry weight of the composition that is subjected to emulsification. Or in other words, wherein less than 4 wt %, more preferably less than 3 wt %, most preferably less than 1 wt % protein based on total dry weight in the spray-dried composition is present in the first aqueous phase and in the emulsified O/W composition. The protein is added to the emulsified O/W composition after emulsification, more preferably is added to the emulsified O/W composition before being subjected to pasteurization and spray-drying. Thus, in an embodiment wherein the nutritional composition comprises protein, preferably less than 4 wt % protein, preferably less than 3 wt %, more preferably less than 1 wt % protein is present in the lipid-containing particle comprising a lipid-containing core and OSA starch coating surrounding the lipid core based on total protein weight in the nutritional composition. In a further preferred embodiment, less than 10 wt % digestible carbohydrates, based on dry weight of the emulsified O/W composition, is present during emulsification, preferably less than 4 wt %. Alternatively worded, the emulsified O/W composition preferably comprises less than 10 wt % digestible carbohydrates, more preferably less than 4 wt %, based on the total dry weight of the emulsified O/W composition.

The method according to the invention thus preferably applies a split stream process wherein lipid and protein are processed separately such that an emulsified O/W composition is obtained which is solely stabilised by the selected emulsifier and avoids the formation of a mixed interface containing proteins and the emulsifier. The inventors have surprisingly found that such an emulsification allows to obtain a more stable composition upon reconstitution and an improved design of the interface with lipid comprising negatively charged OSA starch and is optimised to achieve a targeted performance during digestion (e.g. the composition remains stable during gastric digestion in the acid environment of the stomach). Preferably, the dry weight ratio of OSA starch to lipid is in the range from 1:3 to 1:5, more preferably from 1:3.5 to 1:4.5 during emulsification.

Dependent on the type of OSA starch used, emulsification of lipid is performed at a neutral or acidic pH. For emulsifying properties of OSA starch, steric repulsion or hindrance is important. Emulsification and the provision of a stabilized emulsion by proteins is largely dependent on the electrostatic repulsive forces and thereby rely on pH. Hence, emulsification at pH values closer to the iso-electric point of the protein may be inadequate leading to unstable emulsions. OSA starch advantageously is able to emulsify at lower pH values and to remain stable at acidic pH values thereby more resistant to the acidic environment in the stomach. In a preferred embodiment, emulsification of lipid with a first aqueous phase and OSA starch is performed at a pH in the range of 3-6.5. More preferably, emulsification of lipid with a first aqueous phase and OSA starch is performed at a pH in the range of 3-5.5, preferably in the range of 3.5 to 5. Herein, the pH range relates to the pH of the first aqueous phase, thus, preferably, the first aqueous phase has a pH in the range of 3-6.5. More preferably, the first aqueous phase has a pH in the range of 3-5.5, more preferably in the range of 3.5 to 5.

Preferably, OSA starch is dissolved in water and is added either separately to the lipid and the first aqueous phase or it can be added to the first aqueous phase before emulsification. Preferably, OSA starch is dissolved in the first aqueous phase. In a further preferred embodiment, the first aqueous phase comprises at least water and optionally further comprises one or more of the OSA starch, an encapsulating agent, water-soluble vitamins, water-soluble minerals, flavouring agents, colourants and/or sweeteners. In a further preferred embodiment, the first aqueous phase comprises less than 10 wt %, preferably less than 4 wt % of the digestible carbohydrates based on total dry weight in the nutritional spray-dried composition obtained by the method according to the invention. In a preferred embodiment, emulsification of lipid with OSA starch and the first aqueous phase is performed via homogenization, preferably at a temperature between 5° and 75° C.

In a preferred embodiment, protein is mixed with the emulsified O/W composition by adding protein to a second aqueous phase and combining the emulsified O/W composition with the second aqueous phase before spray-drying and/or protein is added as a (dry-)blend to the spray-dried composition in the method according to the invention. Adding protein as (dry-)blend to the spray-dried composition depends on the microbiological quality of the protein powder and hence when protein has been treated separately and is microbiologically safe, it is preferred that protein is added to the spray-dried composition. This can save the protein from being subjected to undesirable heat conditions. In an especially preferred embodiment, protein is mixed with the emulsified O/W composition by adding protein to a second aqueous phase before subjecting the composition to a spray-drying step, and optionally also before a pasteurization step. In a further preferred embodiment, protein is added to the second aqueous phase by means of homogenization.

Preferably, the second aqueous phase comprises at least water and optionally further comprises one or more of protein, an encapsulating agent, and further water-soluble components selected from water-soluble vitamins, water-soluble minerals, flavouring agents, colourants and/or sweeteners, more preferably the second aqueous phase comprises water and protein. In a further preferred embodiment, the second aqueous phase comprises less than 4 wt % of the digestible carbohydrates based on total dry weight of the digestible carbohydrates in the spray-dried nutritional composition. In a preferred embodiment of the method, the second aqueous phase is combined with the emulsified O/W composition by means of homogenization.

In a preferred embodiment, further ingredients can be added to the emulsified O/W composition after emulsification, e.g. by means of blending and/or homogenization such as one or more selected from an encapsulating agent, further vitamins, minerals, flavouring agents, colourants and/or sweeteners. The further ingredients added to the emulsified O/W composition can comprise lipid-soluble and/or water-soluble components, preferably the further ingredients are water-soluble to prevent an increase in the free lipid content in the composition. If lipid-soluble components are added to the emulsified O/W composition after emulsification of lipid, it is preferred that the total amount of the lipid-soluble components is less than 10 wt %, more preferably less than 8 wt %, most preferably less than 4 wt % based on total dry weight of the emulsified O/W composition that subjected to spray-drying. In a further preferred embodiment, the emulsified O/W composition comprises less than 10 wt %, more preferably less than 4 wt % digestible carbohydrates based on total dry weight of the emulsified O/W composition. This is to prevent gelation of the digestible carbohydrates during spray-drying. Hence, in a preferred embodiment, if digestible carbohydrates are present, these are preferably dry-blended into the spray-dried composition.

In a preferred embodiment, the encapsulating agent is added to the emulsified O/W composition prior to spray-drying, more preferably the encapsulating agent is added to the first aqueous phase and/or to the second aqueous phase, and/or added as a further ingredient to the emulsified O/W composition that subjected to spray-drying. In a preferred embodiment, coating with the encapsulating agent is performed during spray-drying in the method according to the invention, more preferably, during spray-drying the encapsulating agent coats the lipid particles in the emulsified O/W composition. In a further preferred embodiment, the encapsulating agent coats the OSA starch-stabilized lipid-containing particles.

It is preferred that the method according to the invention comprises a pasteurization step, preferably before a spray-drying step. Preferably, pasteurization of the emulsified O/W composition is performed after addition of an optional second aqueous phase. In a further preferred embodiment, pasteurization is performed via direct heating (e.g. direct steam injection or steam infusion) or via indirect heating (e.g. tubular pasteurization, plate heat exchanger or retorting).

In a further preferred embodiment, the emulsified O/W composition, optionally further comprising the second aqueous phase, is subjected to a spray-drying step to obtain a spray-dried composition. In this regard, any spray-drying apparatus suitable for drying food compositions may be used. In one embodiment, the spray-dried composition as used herein is a nutritional composition but considered not a nutritionally complete composition as further ingredients are added to the spray-dried composition by means of dry-blending or mixing the further ingredients to the spray-dried composition. In an alternative embodiment, the spray-dried composition is a nutritionally complete composition and any further ingredients such as micronutrients are added to the first or second aqueous phase or added to the lipid before emulsification. The spray-dried composition is preferably considered gastric stable. In other words, the spray-dried composition comprises the OSA starch-stabilized emulsion according to the invention.

Furthermore, it is preferred that in the method according to the invention the carbohydrates available for digestion are excluded from wet-phase processing. Preferably, the digestible carbohydrates are added to the spray-dried composition in a (dry-)blending step to obtain the nutritional composition according to the invention. This is preferably done to avoid gelation and preserve the digestible carbohydrates in their original (native) structure. The preservation of the digestible carbohydrate structure allows to achieve a slower carbohydrate digestion, which together with a gastric-stable emulsion ensures slow and more sustained release of glucose during digestion. Consequently, it is preferred that the digestible carbohydrates are added to the spray-dried composition in a (dry-)blend, optionally with one or more additional dry ingredients such as acidity regulators, flavours, colorants and/or sweeteners. In a preferred embodiment, at least 80 wt %, more preferably at least 85 wt %, most preferably at least 96 wt % of the digestible carbohydrates based on total dry weight of the digestible carbohydrates in the final nutritional composition is added to the spray-dried composition in a (dry-)blend optionally with one or more additional dry ingredients such as acidity regulators, flavours, colorants and/or sweeteners, preferably the food-grade pH adjusting agent.

### Emulsified Oil-In-Water (O/W) Composition Obtainable by the Method According to the Invention

In a further embodiment the invention relates to an emulsified O/W composition obtainable by the method according to the invention. Preferably, the emulsified O/W composition obtainable by the method according to the invention comprises lipid, OSA starch and optionally an encapsulating agent. Optionally, the emulsified O/W composition comprises protein that is added to the emulsified O/W composition in second aqueous phase. In a preferred embodiment, the emulsified O/W composition according to the invention comprises lipid-containing particles, said particles having a core comprising lipid and wherein the core is coated with OSA starch and the lipid core and OSA starch coating surrounding the lipid core are essentially free from protein. Preferably, wherein the coating of OSA starch prevents the lipid core from making contact with an outer layer comprising protein and/or prevents the lipid core from making contact with protein that is adsorbed onto or forms a separate layer onto the OSA starch coating. Herein, if protein is present, preferably less than 4 wt % protein is present in the lipid core and OSA starch coating, based on total dry weight of the emulsified O/W composition.

The emulsified O/W composition as used herein is not a nutritionally complete composition, i.e. the O/W composition does not contain the essential nutrients including essential amino acids, essential fatty acids, vitamins, and minerals, or adequate calories to meet the energy needs of the individual, based on their age, sex, activity level, and other factors, or balanced macronutrient in terms of digestible carbohydrates, proteins, and fats—to support various bodily functions.

The emulsified O/W composition is considered gastric stable. In other words, the emulsified O/W composition comprises the O/W emulsion stabilized with OSA starch according to the invention. It is preferred that the emulsified O/W composition does not comprise digestible carbohydrates to prevent gelation of the emulsified O/W composition during spray-drying to obtain the spray-dried composition. Preferably, the emulsified O/W composition comprises less than 10 wt %, more preferably less than 4 wt % digestible carbohydrates based on the total dry weight of the composition.

### OSA Starch

The emulsifier in the present invention is an octenyl succinic anhydride substituted starch compound (herein also referred to as “OSA starch”) and is considered stable under gastric conditions. OSA starches are often available as a sodium salt. The OSA starch is preferably a food approved OSA starch (International Numbering System codex E1450). In an embodiment the OSA starch is derived from a waxy maize starch, tapioca starch, rice starch, potato starch, wheat starch, starch from other plant origin substituted with octenylsuccinic acid groups, such as other maize or other crops origin. Reference is made to “Structure and physicochemical properties of octenyl succinic anhydride modified starches: A 5 review” by M. C. Sweedman et al., in Carbohydrate Polymers 92 (2013) 905-920 that discloses several OSA starches that may find use in the present invention. Preferably, the OSA starch as used herein is non-gelatinized, more preferably the OSA starch comprises a granular structure and has not undergone a gelatinization process. The use of additional emulsifiers in making the O/W emulsion and the O/W composition are not excluded by the present invention, but preferably only one or more OSA starch compounds are employed. In this regard, additional emulsifiers should be gastric-stable in an acidic environment as in the stomach as defined here above or in other words, the additional emulsifiers should be stable under gastric conditions in the stomach as will be defined further below.

Preferably the dry weight ratio OSA starch to lipid present in the emulsified O/W composition is from 1:3.5 to 1:4.5. Preferably, the same dry weight ratios of OSA starch to lipid applies in the final nutritional composition. Working at these conditions, the lipid core is optimally coated. In this regard, if the amount of OSA starch is lower or if the weight ratio OSA starch to lipid is lower, the O/W emulsion may become less stable and more prone to rapidly coalesce in a gastric environment. Consequently, a lipid layer may be formed on the top of an aqueous phase, instead or in addition to the desired homogenous dispersion of lipid. On the other hand, if the amount of OSA starch is higher than above, or if the weight ratio OSA starch to lipid is higher than above, more free OSA starch not covering lipid globules/the lipid core may be present in the O/W composition (and in final nutritional composition), which can cause side-effects in subjects suffering from carbohydrate-related metabolic disorders, such as gas and bloating.

The amount of OSA starch in the spray-dried nutritional composition is preferably in the range of 2-20 wt % based on total dry weight of the spray-dried composition, more preferably in the range of 3-16 wt %, most preferably in the range of 4-14 wt % based on total dry weight of the spray-dried composition. In a further preferred embodiment, the amount of OSA starch is preferably in the range of 2 to 6 wt % based on total dry weight of the nutritional composition, more preferably in the range of 2.5 to 5 wt %, most preferably in the range of 3-4 wt %.

The OSA starch serves as an emulsifier due to its amphiphilic nature. The OSA starch preferably has a low viscosity profile for high concentration of lipid in emulsions to obtain low viscosity emulsions and ensuring an effective emulsification. Thus, preferably, the OSA starch has a viscosity of 100-400 mPas at 70 deg C. at 1000 1/s and 100-600 mPas at 70 deg C. at 100 1/s. The viscosity can be determined using any suitable method for measuring viscosity, preferably viscosity is measured as according to ISO 3219-1 and 2:2021. The benefit of this particular OSA-starch is that it results in an increase of the viscosity of the aqueous phase relative to the viscosity of the lipid phase in an O/W emulsion, thereby reducing the mobility of lipid globules and reducing the occurrence of lipid coalescence and providing a homogeneous emulsion of lipid and water. This reduction in lipid coalescence in the OSA starch-stabilized emulsion results in a gastric-stable nutritional composition with a lower free lipid content.

### Lipid

Preferably, the amount of lipid in the spray-dried composition is at least 10 wt % based on dry weight of the spray-dried composition. Preferably, lipid in the spray-dried composition is in the range of 10-55 wt % based on total dry weight of the spray-dried composition, more preferably in the range of 15-50 wt %, most preferably in the range of 20-45 wt % based on total dry weight of the spray-dried composition. The lipid in the O/W composition has been emulsified with OSA starch before being subjected to spray-drying to obtain the spray-dried composition. Hence, preferably the emulsified O/W composition comprises the same amount of lipid as in the spray-dried composition. In a further preferred embodiment, the final nutritional composition comprises at least 10 wt % lipid based on dry weight of the final nutritional composition. Preferably the final nutritional composition comprises 10 to 30 wt % lipid, more preferably 11-20 wt % lipid based on dry weight of the final nutritional composition.

The lipid in the final nutritional composition provides for between 15 and 40 en %, preferably between 20 and 35 en %, most preferably between 25 and 30 en % based on total energy content of the final nutritional composition.

Naturally, the lipid is an edible fat or oil. Edible oils used in the context of the invention may be obtained from natural sources, for example plants, microbes and marine sources, algae oil. Suitable plant sources include, but are not limited to, flaxseed, walnuts, sunflower seeds, canola oil, safflower oil, soy, wheat germ, leafy green plants such as kale, spinach and parsley, and corn oil. The edible oil may be present in a purified form and/or in the form of an extract from a suitable source. The lipid ingredient may be an edible oil, but may also be a mixture of edible fat or oils, such as a mixture of edible oils from two or more sources.

The lipid of the present invention preferably comprises (tri)glycerides. Triglycerides comprise a glycerol molecule to which, via ester bonds, three fatty acid residues are attached, which may be the same or different, and which are generally chosen from saturated and unsaturated fatty acids containing 6 to 26 carbon atoms, including but not limited to linoleic acid (18:2 n6) (LA), alpha-linolenic acid (18:3 n3) (ALA), oleic acid (C18:1), palmitic acid (16:0) and/or stearic acid (C18:0). Such fatty acid triglycerides may differ in the fatty acid residues that are present and/or in the respective position(s) of the fatty acid residues, e.g. in the sn-1, -2 and/or -3 position.

It is advantageous to use vegetable lipid as part of the lipid ingredient as vegetable lipid is digested into free fatty acids and glycerol in the small intestines and only to a smaller extent in the stomach. Before uptake of free fatty acids into the bloodstream, the free fatty acids exert a physiological effect, which affects the gastrointestinal motor and secretory activity mediated the sensing-mechanism in the small intestinal mucosa by the free fatty acids. Without being bound to any theories, it is hypothesized that the free fatty acids bind to G-coupled receptors (GPR) located in the enteroendocrine cells (EEC) in the small intestinal epithelia thereby affecting satiety and gastrointestinal motor activity by the release of the peptides, such as CCK, GLP-1 and peptide YY. Increased expression of these peptides may result in a decreased gastric emptying. Especially the presence of long chain free fatty acids (with a fatty acid chain of 13 or more carbon atoms) in the intestinal phase may increase plasma CCK levels, whilst unsaturated long chain free fatty acids may promote the secretion of GLP-1. A decreased gastric emptying rate, and retention of nutritional food in the stomach ensures a gradual digestion in the gastro-intestinal tract.

Thus in a preferred embodiment, the lipid comprises at least 50 wt % vegetable lipid based on total dry weight of the lipid, more preferably at least 75 wt %, even more preferably at least 85 wt %, most preferably at least 95 wt %. In a further preferred embodiment, vegetable lipid comprises sunflower oil, more preferably sunflower oil is the only vegetable lipid in the emulsified O/W composition, in the spray-dried composition and in the final nutritional composition. In a preferred embodiment, the lipid comprises 4-8 wt % palmitic acid, 3-6 wt % stearic acid, 16-22 wt % oleic acid, 60-70 wt % LA and 0.3-0.8 wt % ALA based on total dry weight of the lipid in the nutritional composition.

It is preferred that the lipid in the emulsified O/W composition, in the spray-dried composition and in the final nutritional composition is present in lipid-containing particles comprising lipid and OSA starch, wherein the lipid-containing particles have a core comprising lipid and wherein the core is coated with OSA starch and wherein the lipid core and OSA starch coating surrounding the lipid core are essentially free from protein.

In this regard, the coating with OSA starch does not imply that it must fully cover the surface of the lipid core but that it means that the resulting emulsified O/W composition, the spray-dried composition and the final nutritional composition are gastric-stable comprising the gastric-stable emulsion and can be reconstituted into a gastric-stable emulsion because the emulsification of lipid with OSA starch is performed essentially free from protein as defined herein, preferably wherein less than 4 wt %, more preferably less than 3 wt %, most preferably less than 1 wt % protein is present during emulsification based on total dry weight in the emulsified O/W composition and/or the spray-dried composition and/or the final nutritional composition and/or wherein the lipid-containing particles have a core comprising lipid and said core is coated with OSA starch, wherein said lipid core and OSA starch coating comprise less than 4 wt % protein, more preferably less than 3 wt %, most preferably less than 1 wt % protein based on total dry weight in the emulsified O/W composition and/or the spray-dried composition and/or the nutritional composition.

The stability is considered to be attributed to the steric repulsion which is generated by the branched structure of OSA starch, thereby preventing other components to cover the lipid core. Also, the emulsification of lipid is in presence of OSA starch but in substantial absence of protein, preferably less than 4 wt %, more preferably less than 3 wt %, most preferably less than 1 wt % protein is present based on total dry weight of the O/W composition that is subjected to emulsification. These advantageous effects can be achieved when OSA starch is added to the lipid prior to or during emulsification wherein the dry weight ratio of OSA starch to lipid is in the range from 1:3 to 1:5, and preferably the dry weight ratio OSA starch to lipid is from 1:3.5 to 1:4.5.

The emulsified O/W composition and/or the spray-dried composition and/or final nutritional composition are advantageously low in free lipid content. In the context of the invention, ‘free lipid content’ is understood as all lipid particles not coated with OSA starch and thereby susceptible of oxidation. Thus, preferably at least 90 wt % lipid based on total lipid weight in the final nutritional composition is subjected to emulsification with OSA starch, more preferably at least 95 wt %, most preferably at least 98 wt %. Free lipids oxidize in the O/W composition and in final nutritional composition thereby reducing palatability of the final product. In addition, when not coated by OSA starch, free lipids affect digestibility and the gastric emptying rate. Hence, it is preferred that the free lipid content, i.e. all lipid not coated with OSA starch, is less than 10 wt % lipid based on total lipid weight in the emulsified O/W composition and/or the spray-dried composition and/or final nutritional composition, preferably less than 8 wt %, most preferably less than 4 wt % lipid based on total lipid weight in the final nutritional composition.

In the context of the present invention, the free lipid content is determined by the method as described in “Determination of Free Fat on the Surface of Milk Powder Particles”, Analytical Method for Dry Milk Products, NS NIRO ATOMIZER (1978). Samples are prepared by finely grinding the powder with a cutter to avoid grinding it down entirely. Subsequently, the powder is passed through a 32 mesh sieve after which the free lipid content in the samples is measured using NS NIRO ATOMIZER. The content of free lipid determined by this method is represented by wt % of the lipid extracted with carbon tetrachloride under shaking at constant rate within the prescribed time.

The lipid fraction may also comprise non-vegetable lipids. Non-vegetable lipids may include milk fat, milk derived lipids as a preferred source of phospholipids, fish, marine and/or microbial oils as source of long chain polyunsaturated fatty acids. Preferably the lipid comprises fish oil. Preferably the lipid comprises EPA, DPA and/or DHA, more preferably DHA and EPA.

The content of omega-3 LC-PUFA, more preferably DHA and EPA, preferably does not exceed 10 wt % of the total fatty acid weight, preferably does not exceed 7 wt %, even more preferably does not exceed 3 wt % of the total lipid content. Preferably the present composition comprises at least 0.15 wt %, preferably at least 0.35 wt %, more preferably at least 0.65 wt % omega-3 LC-PUFA, more preferably DHA and EPA, of the total lipid content. In one embodiment, the lipid fraction comprises at least 0.15 wt % omega-3 LC-PUFA based on total fatty acids selected from the group consisting of DHA, EPA, and DPA, more preferably DHA and EPA.

Furthermore, the lipid fraction preferably comprises one or more additional components that are lipid-compatible (hydrophobic) like lipid-compatible vitamins, such as vitamins A, D, and E. The amounts of these vitamins are compliant with regulations for food for special medical purposes such as Food for Special Medical Purposes (FSMP) directive 1999/21/EC of 25 Mar. 1999. In this regard, the amount of vitamins and minerals is dependent on the age of the consumer and thus is preferably in the range from 20 to 100% of the daily recommended intake, more preferably in the range from 50 to 75%.

### Digestible Carbohydrates

The nutritional spray-dried composition preferably comprises less than 10 wt % digestible carbohydrates, more preferably less than 4 wt % based on total dry weight of the nutritional spray-dried composition. The digestible carbohydrates are preferably dry-blended into the nutritional spray-dried composition. After dry-blending, the nutritional composition preferably comprises 25-75 wt % digestible carbohydrates based on total dry weight of the nutritional composition, more preferably 30-70 wt %, most preferably 35-65 wt %.

Preferably the nutritional composition comprises digestible carbohydrates such as modified starches and/or native starches. In the context of the invention, starch digestibility is classified into rapidly digestible starch (RDS), slowly digestible starch (SDS) and resistant starch (RS) fractions based on the method described by Englyst et al., Classification and measurement of nutritionally important starch fractions, Eur J Clin Nutr, 1992, 46, Suppl 2, S33-50. The relative amount of each fraction is based on the amount of glucose released during in vitro digestion. In the context of the invention, RDS is defined herein as starch (or carbohydrate) which is digested within 20 minutes of in vitro intestinal digestion, SDS is defined herein as starch or carbohydrate which is digested within 20 to 120 minutes of in vitro intestinal digestion while RS is defined herein as starch or carbohydrate which is not digested after 120 minutes of in vitro intestinal digestion.

In the context of the invention, the digestible carbohydrates are preferably digestible starches, or also referred to as digestible carbohydrates and are available for digestion and glycolytic breakdown. In this regard, it should be noted that one source of digestible carbohydrates may comprise a fraction of SDS, RDS and RS at once. For example, uncooked corn starch which can be used as carbohydrate source for administration to patients suffering from carbohydrate-related metabolic disorders comprises a fraction of RDS, SDS and RS.

In a preferred embodiment, the source of digestible carbohydrates, more preferably the source of SDS, is selected from one or more of starch-rich foods such as grains, legumes, roots and tubers. In a further preferred embodiment, the digestible carbohydrates are selected from maize, corn, wheat, potato, rice, cassava and/or tapioca, more preferably the digestible carbohydrates comprises maize starch. It is preferred that maize starch is the predominant digestible carbohydrate source, more preferably maize starch is the only digestible carbohydrate source. Maize starch breaks down into glucose during digestion and comprises a large fraction of SDS. Different starch and maize starch ingredients can differ in their resistance towards gastrointestinal digestion. High fractions of RDS in a digestible carbohydrate source may yield a rapid increase in postprandial plasma glucose, contributing to a peak in blood glucose levels, stimulating insulin secretion. Increased demand for insulin by elevated plasma glucose levels can be associated with an increased risk on developing diabetes type 2 (T2DM). Besides a risk on developing T2DM, increased plasma glucose levels can also be associated with the development of other chronic diseases, cardiovascular diseases by oxidative stress and obesity. SDS starches may yield a slow increase of postprandial plasma glucose levels and sustained release of glucose preventing an initial period of highly increased plasma glucose levels. The SDS starch fraction may support prolonged normoglycemia in subjects in need thereof such as GSD patients. A too large fraction of RS starches can cause gastrointestinal discomfort, including bloating and nausea.

It is preferred that the digestible carbohydrates comprise native starch or starch processed such that the SDS fraction has been increased via means of retrogradation, annealing or other processes to modify starch to increase the SDS fraction. In the context of the invention, ‘native’ starch means the starch is not treated or only minimally treated, but that in all cases the starch is at least uncooked and thereby comprises higher fractions of SDS. The original granule and crystal structure in the native starch is still present as it was in the grain thereby the accessibility of the starch for the amylase enzyme is more limited, hence leading to slower digestion of the native starch. The “nativity” of starch refers to its inherent properties, including its structure and behavior when in its natural, unmodified state. One common way to assess the nativity of starch is through the use of X-ray diffraction analysis.

In a preferred embodiment, the digestible carbohydrates comprises native starch, more preferably native maize starch. If a starch is not native, the original granule and crystal structure is most likely not present unless such a structure has been modified deliberately in such a way by recrystallization during cooling after heating, i.e. by means of retrogradation. Thus, in alternative embodiment, the digestible carbohydrates may comprise modified starch by means of recrystallization and/or retrogradation, preferably modified maize starch.

It is preferred that the digestible carbohydrates, present in the nutritional composition, comprise at least 55 wt % SDS based on total dry weight of the digestible carbohydrates, more preferably at least 60 wt % SDS, most preferably at least 65 wt % SDS. Preferably, the digestible carbohydrates comprise less than 20 wt % of RS based on total dry weight of the digestible carbohydrates, more preferably less than 15 wt %, most preferably less than 13 wt % of RS based on total dry weight of the digestible carbohydrates. The amount of RDS as part of the digestible carbohydrates is preferably kept low. Preferably, the digestible carbohydrates comprise less than 45 wt % RDS based on total dry weight of the digestible carbohydrates, more preferably less than 40 wt %, most preferably less than 35 wt %. In further preferred embodiment, the dry weight ratio of SDS to RDS is in the range of 1:1 to 2:1, preferably in the range of 1.3:1 to 2:1, more preferably in the range of 1.5:1 to 2:1.

Furthermore, OSA starch is considered as emulsifier in the context of the invention while resistant maltodextrin is considered the encapsulating agent and are not taken into account as part of the weight of the digestible carbohydrates.

In a preferred embodiment, the digestible carbohydrates are added after emulsification and spray-drying of the O/W composition which comprises OSA starch and lipid. It is preferred that the digestible carbohydrates comprising the slowly digestible starches are added to the spray-dried composition by means of a dry-blend to prevent gelation of the carbohydrates in wet-phase processing.

The digestible carbohydrates in the final nutritional composition provides for between 40 and 65 en %, preferably between 45 and 60 en %, most preferably between 50 and 55 en % based on total energy content of the final nutritional composition.

### Protein

It is essential that only very limited amount of protein is present during the emulsification of lipid with OSA starch to prevent interference with migration of OSA starch to the emulsion interface and/or positioning of protein on the emulsion interface instead of OSA starch. The inventors have surprisingly found that presence of protein or other surface-active components at the emulsion interface are less resilient to the gastric environment and can destabilize the emulsion during gastric digestion. Hence emulsification of lipid with OSA starch without the presence or limited presence of protein allows to obtain a more stable emulsion upon reconstitution and an improved coating of OSA starch around lipid globules/lipid core. In this manner, the nutritional composition upon reconstitution remains stable during gastric digestion. Hence, preferably, if any, protein is added to the emulsified O/W composition before pasteurization and spray-drying and/or added as a dry-blend after spray-drying.

In a preferred embodiment, the optional protein is added to the emulsified O/W composition before being subjected to pasteurization and spray-drying. Preferably, the optional protein is present in a second aqueous phase which is added to the emulsified O/W composition comprising lipid and OSA starch.

If protein is present during emulsification, it is preferably present in an amount less than 4 wt %, more preferably less than 3 wt %, most preferably less than 1 wt % based on total dry weight of the emulsified O/W composition. Or in other words, wherein less than 4 wt % protein, preferably less than 3 wt %, more preferably less than 1 wt % based on total protein weight of the spray-dried and/or the nutritional composition is present in the first aqueous phase and in the emulsified O/W composition. Regarding the nutritional composition, less than 4 wt % protein, preferably less than 3 wt %, more preferably less than 1 wt % protein is present in the lipid core and its OSA starch coating altogether, based on total dry weight in the nutritional composition. Preferably no protein is added prior to and during emulsification of lipid with an aqueous phase and OSA starch. As a consequence, with no or low protein the lipid core a gastric-stable emulsion is obtained, which yields less degradation in the acidic environment of the stomach, and slower release of nutrients.

The amount of protein, if present, in the spray-dried composition, is preferably in the range of 20-70 wt % based on total dry weight of the spray-dried composition, more preferably in the range of 25-65 wt %, most preferably in the range of 30-60 wt % based on total dry weight of the spray-dried composition.

The nutritional composition according to the invention optionally comprises protein to obtain a nutritionally more complete product. Protein digestion may stimulate insulin and incretin hormone secretion and supports a slow gastric emptying thereby supporting a slow release of carbohydrates in the small intestine. Optionally, the nutritional composition comprises 10-35 wt % protein based on total dry weight of the final nutritional composition, more preferably 12-30 wt %, most preferably 15-27 wt % based on dry weight of the final nutritional composition.

If present in the final nutritional composition, the protein in the final nutritional composition provides for between 5 and 35 en % based on total energy content, preferably between 10 and 30 en %, most preferably between 15 and 25 en % based on total energy content of the final nutritional composition. In the current diet of GSD patients the main energy source comes from carbohydrates. However, optionally, protein can be used as metabolic compound and energy supplier in the gluconeogenesis pathway and as a building block for muscle gain.

Suitable examples of proteins that beneficially can be added to either the emulsified O/W composition and/or to the spray-dried composition are protein derived from dairy, plant, or microbial sources, preferably one or more selected from whey protein and/or proteins selected from pulse protein, oil seed proteins, cereal proteins and/or mycoproteins, more preferably selected from potato, grains such as wheat and/or rice, pea, soy, or beet and mixtures thereof. Optionally, the proteins may be completely or partly hydrolysed or comprise free amino acids. Preferably, the protein source involves intact protein or protein that is at least 90% intact. Mixtures of several protein sources and mixtures of intact and hydrolysed protein are also possible. Most preferably, the protein comprises intact whey protein.

A measure for the extent of hydrolysation of the whey protein is the “degree of hydrolysation” (DH). The DH is defined as the percentage of the total number of peptide bonds in a protein that has been cleaved during hydrolysis and therefore serves as a measure how intact whey protein is. The DH of a protein may e.g. be determined by the trinitrobenzenesulphonic acid (TNBS) procedure, as known in the art (Adler-Nissen, J. Agr. Food Chem. 1979, 27(6), 1256). When whey protein is subjected to a hydrolysis process, the source of whey protein may already comprise a certain (small) amount of peptide fractions, before being subjected to the hydrolysis process. The values for the degree of hydrolysation as described herein are corrected for this presence of peptide-fractions in the whey protein source, in other words, the values for the DH are corrected for the natural DH of whey protein. Herein, the DH thus relates to the additional hydrolysation that was obtained via the intentional hydrolysis process. When the final nutritional composition comprises hydrolysed whey protein, it preferably has a degree of hydrolysation of 1-75%, preferably in the range of 5 to 60%, more preferably in the range of 10 to 50%. As described above, the degree of hydrolysation as used herein is corrected for the natural degree of hydrolysation of the whey protein source, i.e. the whey protein that was used for the preparation of the hydrolysed whey protein. In a preferred embodiment, the optional protein comprises intact whey protein which has not been subjected to a hydrolysis process, i.e. has not been subjected an additional hydrolysation that was obtained via an intentional hydrolysis process

In an alternative embodiment, protein is produced by or obtainable by precision fermentation. In case the protein source has poor agglomeration properties and is thereby less suitable to spray-dry, such as protein hydrolysates and free amino acids, it is preferred that these hydrolysates and/or amino acids are added in a dry-blending step to the gastric stable spray-dried composition.

### Encapsulating Agent—Resistant Maltodextrin

In a preferred embodiment, the spray-dried composition and the final nutritional composition comprises an encapsulating agent. The role of the encapsulating agent is to stabilize the composition during the (spray-)drying process and to protect the composition and particularly the lipids therein from degradation or undesirable interactions with the surrounding environment. In a preferred embodiment, the encapsulating agent coats the lipids and any further ingredients added to the composition prior to spray-drying, more preferably the encapsulating agent coats the lipid-containing particles and thereby forms an additional coating over the lipid coated with OSA starch. In a further preferred embodiment, when other further ingredients are added to the composition before spray-drying, preferably protein, the encapsulating agent also coats these further ingredients, preferably the encapsulating agent coats protein. The encapsulating agent helps to form a protective barrier around the particles in the spray-dried composition, allowing it to retain its properties and functionality in the powdered product. The encapsulating agent or spray-drying agent for microencapsulation of the composition is necessary to achieve high encapsulation efficiency, high drying efficiency, and high stability of the microcapsules. Maltodextrins or other rapidly digestible carbohydrates are often used as a spray-drying agent to produce a nutritional powder product by reducing the thermoplasticity and hygroscopicity as well as the stickiness and product deposition.

In a preferred embodiment, the encapsulating agent is a food-grade encapsulating agent, meaning that the encapsulating agent is safe for consumption, more preferably the encapsulating agent is selected from maltodextrins, glucose syrup, trehalose, maltose, amylose and/or amylopectin, even more preferably the encapsulating agent is a maltodextrin, most preferably a resistant maltodextrin. Herein referred to, the encapsulating agent is not OSA starch which serves as an emulsifier coating the lipid. Alternatively worded, the encapsulating agent serves as a second encapsulating agent forming an additional coating over the lipid-containing particles and optionally further ingredients, such as protein. If such a second coating is applied onto the lipid containing particles, the encapsulating agent preferably does not comprise OSA starch.

Alternatively, the encapsulating agent has a glycaemic index in the range of 0 to 50, preferably in the range of 0 to 25, more preferably the encapsulating agent comprises resistant maltodextrin. Herein, the glycaemic index is a commonly used term assigned to foods, in particular to sugars and carbohydrates, which ranges from 0 to 100. Herein, a glycaemic index of 0 means said food, sugar, carbohydrate or the like does not raise blood sugar levels at all. A glycaemic index of 100 raises blood sugar levels almost immediately and this value is typically assigned to pure glucose.

The final nutritional composition and/or the spray-dried composition according to the invention preferably comprises resistant maltodextrin, in order to facilitate drying and coating of the OSA-starch stabilized emulsion. Resistant maltodextrins avoid rapid carbohydrate digestion seen in the use of conventional maltodextrins as processing aids for encapsulation of emulsions, because these avoid the glucose release or ‘peak’ seen after consumption of a meal or product which is an undesired effect in the patient groups considered herein. Resistant maltodextrin is a soluble fibre with a higher digestive tolerance and, as a result, passes through to the large intestine relatively intact. Examples of resistant maltodextrins on the market are Promitor® Soluble Fiber (from Tate&Lyle) and Fibersol®-2 (from Fibersol®).

In a preferred embodiment, the emulsified O/W composition and/or the spray-dried composition comprises 2-24 wt % of an encapsulating agent based on total dry weight of the composition obtainable by the method according to the invention, more preferably 3-22 wt %, most preferably 5-20 wt %. In a preferred embodiment, the final nutritional composition comprises 2 to 15 wt %, preferably 2.5 to 12 wt %, most preferably 3 to 9 wt % of an encapsulating agent based on total dry weight of the final nutritional composition.

### Further Components

The nutritional (spray dried) composition according to the invention preferably comprises further components to obtain a nutritionally complete composition. In subjects essentially adhering to e.g. single sources of nutrition, it is essential that the nutritional requirements are met to support development, and to maintain essential biological processes, respectively. For example, GSD patients consume relatively high amounts of carbohydrates as part of the dietary treatment and restrictions of others foods such as lactose and fructose, these patients are at a higher risk of a certain nutrient deficiency. For this reason, the bio-accessibility and bio-availability of minerals and trace elements from nutritional products should be high.

Therefore, the nutritional spray dried composition of the invention is preferably supplemented with additional ingredients, such as further proteins (protein hydrolysates and/or amino acids), further lipids, further carbohydrates (digestible and non-digestible), vitamins, biotics (one or more of probiotics, prebiotics and/or postbiotics), further trace elements or other micronutrients such as nucleotides. Preferably at least probiotics and/or prebiotics, vitamins and minerals are added to the nutritional composition to provide for a nutritionally complete nutritional composition. Optionally, the nutritional composition of the invention may be further supplemented with acidity regulators such as citric acid and ingredients to adjust organoleptic properties such as flavours, colourants and sweeteners.

The nutritional spray-dried composition according to the invention preferably comprises a food-grade pH adjusting agent in order to lower the pH of the nutritional composition to an acidic value when reconstituted for consumption and when the nutritional composition is in liquid form. The optimal pH for salivary a-amylase enzyme activity is between 6.5 and 7.0 and is considered inactive, or at least significantly less active, at a pH below 5.0. Hence, it is preferred that the nutritional composition has a pH lower than the optimal pH for salivary a-amylase enzyme activity to reduce digestion of carbohydrates, maintaining a more gastric-stable composition, and further slowing down the glycolytic effect in the stomach. In a preferred embodiment, the nutritional composition is intended for reconstitution and comprises a food-grade pH adjusting agent, the pH of the nutritional composition is preferably in the range of 3.0-5.0 when dissolved as 50% w/v solution in water, more preferably in the range of 3.5-4.5, most preferably in the range of 3.8-4.2 when dissolved as 50% w/v solution in water at room temperature. The final nutritional composition preferably comprises 2-14 wt % of a food-grade pH adjusting agent based on total dry weight of the final nutritional composition, more preferably comprises of 4-12 wt %, most preferably comprises 6-10 wt. In a preferred embodiment, the pH adjusting agent comprises an organic acid selected from the group of citric acid, lactic acid, acetic acid, ascorbic acid, malic acid, tartaric acid or a mixture thereof, more preferably the food-grade pH adjusting agent comprises at least citric acid. In the most preferred embodiment, the food-grade pH adjusting agent is citric acid, specifically anhydrous citric acid.

Preferably, further ingredients, including an optional food-grade pH adjusting agent, are added to the emulsified O/W composition prior to spray-drying and are present in the spray-dried composition and/or are dry-blended into the spray-dried composition of the invention.

in an embodiment, dietary fibres are also included in the final nutritional composition. In a preferred embodiment, the dietary fibres comprises fructo-oligosaccharides, galacto-oligosaccharides, galacturonic acid oligosaccharides, more preferably fructo-oligosaccharides and galacto-oligosaccharides. In a preferred embodiment of the method according to the invention, dietary fibres are added to the spray-dried composition in a dry-blending step as fibres are susceptible to gelation which can be avoided as much as possible when these fibres do not undergo spray-drying conditions. Preferably, prebiotics and/or postbiotics are included in the nutritional composition of the invention. In a preferred embodiment of the method according to the invention, prebiotics and/or postbiotics are added to the spray-dried composition in a dry-blending step as subjecting these to spray-drying conditions may deteriorate their activity.

In a preferred embodiment, the final nutritional composition is supplemented with calcium and/or vitamin D as reduced bone mineral density has been reported in ketotic forms of GSD. In a further preferred embodiment, the nutritional composition is further supplemented with vitamin C, vitamin B1, B2, B3, B5 B6, B9, B11 and B12, copper, iodine, iron, magnesium, manganese, molybdenum selenium, zinc, vitamin B2 and vitamin B5. In an embodiment, vitamins and minerals are preferably present in therapeutically effective amounts as used and refers to at least 1, preferably 1.1, more preferably 1.3 times the amount of the recommended daily intake. The RDI is a common reference in the art, for example herewith is referred to the population reference intake (PRI) such as according to the Summary of dietary reference values for the EU population as derived by the European safety Authority as determined in September 2017. The recommended daily intake are defined by authorities such as EFSA or FDA.

Suitable examples of vitamins include vitamins A, B1, B2, B3, B5, B6, B9, B11, B12 C, D E and K. The product and method of the present invention are compatible with lipid-soluble vitamins and water-soluble vitamins and with both water-soluble minerals and water-insoluble minerals. In a preferred embodiment, the vitamins that are more lipid compatible, such as vitamins A, D and E and K, are added to the lipid and so to the spray dryer in the method to prepare the spray-dried composition. In another preferred embodiment the vitamins such as vitamins B and C that are more water compatible, and water-soluble minerals such as calcium and magnesium, are added to the first aqueous phase or to the second aqueous phase or alternatively are added to spray-dried composition in a dry-blending step. In a further preferred embodiment, water-insoluble minerals such as iron, zinc and copper are added to the emulsified O/W composition in an encapsulated form before the composition is subjected to spray-drying or are added to the spray-dried composition by means of a dry-blending step.

In a most preferred embodiment, the final nutritional composition comprises one or more ingredients selected from the group consisting of calcium, copper, iron, magnesium, zinc, vitamin B2, vitamin B5 and vitamin D.

### Nutritional Composition

In a preferred embodiment the final nutritional composition according to the invention is a powder. In an alternative embodiment, the present nutritional composition is a liquid ready-to-feed composition. Preferably the nutritional composition is administered orally or via tube feed.

In a preferred aspect, the final nutritional composition according to the invention may be used as a nutritional product, for example as a nutritional supplement, e.g. as an additive to a normal diet, as a fortifier, to add to a normal diet, or as a complete nutrition, preferably the final nutritional composition is complete nutrition. In a preferred embodiment, the final nutritional composition, being nutritionally complete, is not the exclusive food consumption but preferably the final nutritional composition is consumed as one or more daily doses by subjects in need thereof. For example, GSD patients may use the final nutritional composition during the day before or after a meal while the nutritional composition may also serve as the only intake during the night or before going to sleep.

Preferably, the final nutritional composition comprises between 325 and 500 total calories, more preferably between 350 and 450 total calories per 100 g of the final nutritional composition. The final nutritional composition may contain the daily dosages as defined below in one or more dosage units. In a further preferred embodiment, the final nutritional composition provides for 10 en % to 50 en % of the required daily energy consumption per dosage unit, more preferably for 15 en % to 40 en % of the daily energy consumption per dosage unit.

Preferably the final nutritional composition is a shelf-stable product. A shelf-stable product refers to a packaged food or beverage item that can be stored at room temperature for an extended period without the need for refrigeration or freezing to maintain its quality, safety, and stability. These products are designed to have a long shelf life and remain safe for consumption over an extended period, preferably for at least 30 days. The final nutritional composition of the invention is preferably a packaged product ready for transport and marketing. The composition is thus preferably a solid (typically a powder or tablet, preferably a powder) which is reconstitutable with a liquid to provide a ready-to-feed liquid nutritional product. In a preferred embodiment, when the final nutritional composition is a powder or a tablet, the final nutritional composition comprises carbohydrate-containing particles and lipid-containing particles, wherein said lipid-containing particles are coated with the encapsulating agent and wherein said lipid-containing particles have a core comprising lipid and said core is coated with OSA starch.

Preferably, the final nutritional composition is reconstituted in water. Typically, 50-75 gram of the nutritional composition is dissolved in 100 to 300 ml liquid, preferably water.

The final nutritional composition preferably comprises digestible carbohydrates, lipid, OSA starch, an encapsulating agent and optionally protein and/or a food-grade pH adjusting agent. The final nutritional composition preferably comprises vitamins, minerals and trace elements in amounts sufficient for the subject in need thereof. The amounts of vitamins, minerals and trace elements are preferably compliant with regulations for food for special medical purposes such as Food for Special Medical Purposes (FSMP) directive 1999/21/EC of 25 Mar. 1999. In this regard, the amount of vitamins and minerals is dependent on the age of the consumer and is preferably in the range between 20 and 100% wt % of the daily recommended intake.

Preferably the final nutritional composition comprises an OSA starch to lipid dry weight ratio is in the range from 1:3 to 1:5, more preferably in the range from 1:3.5 to 1:4.5. The ratios preferably also apply to the intermediate compositions before arriving at the final nutritional composition, i.e. the emulsified O/W composition and the (nutritional) spray-dried composition.

In an embodiment according to the invention, the final nutritional composition comprises lipid, OSA starch, an encapsulating agent, digestible carbohydrates and optionally protein, wherein the composition comprises lipid-containing particles which have a core comprising lipid and wherein said lipid core is coated with OSA starch and wherein the lipid core and OSA starch coating comprise less than 4 wt % protein based on total dry weight of the final nutritional composition. Preferably, wherein the coating of OSA starch prevents the lipid core from making contact with an outer layer comprising protein and/or prevents the lipid core from making contact with protein that is adsorbed onto or forms a separate layer onto the OSA starch coating.

Preferably, the final nutritional composition comprises based on total dry weight of the composition:
at least 10 wt % lipid; preferably the lipid comprises sunflower oil;
2-6 wt % OSA starch;
25-75 wt % digestible carbohydrates wherein the digestible carbohydrates comprises at least 90 wt % native maize starch based on total dry weight of the digestible carbohydrates;
2-15 wt % encapsulating agent, wherein the encapsulating agent comprises resistant maltodextrin;
optionally 10-35 wt % protein, preferably intact whey protein.

Preferably, the number of lipid globules, also referred to as lipid-containing particles, in the nutritional composition (and in the emulsified O/W composition and spray-dried composition) not coated with OSA starch is less than 10% based on total number of lipid-containing particles in the nutritional composition (and in the emulsified O/W composition and spray-dried composition), more preferably less than 8%, most preferably less than 4% based on total number of lipid-containing particles.

More preferably, the final nutritional composition comprises lipid-containing particles which are coated with the encapsulating agent and wherein said lipid-containing particles have a core comprising lipid and said core is coated with OSA starch. Optionally, the lipid-containing particles further comprise an outer layer comprising protein and optionally further ingredients. In a preferred embodiment, the lipid-containing particles and/or outer layer surrounding the lipid-containing particles are coated with the encapsulating agent which encapsulating agent-coating prevents the lipid-containing particles and/or the outer layer from making contact with a further ingredient that is adsorbed onto or forms a separate layer onto the encapsulation agent. In a further preferred embodiment, the encapsulating agent-coating is a layer of the encapsulating agent surrounding the lipid-containing particles. Also, in further preferred embodiment, the nutritional composition comprises agglomerates of lipid-containing particles and which agglomerates optionally comprise outer layer comprising protein and further ingredients and wherein the agglomerates are coated with the encapsulating agent.

The use of OSA starch as emulsifier is especially well compatible with nutritional compositions comprising slowly digestible starches (SDS) to prolong gastric emptying and promote slow release of the SDS comprised in the nutritional composition. In a preferred embodiment, the final nutritional composition comprises 25-75 wt % digestible carbohydrates wherein the digestible carbohydrates comprises at least 55 wt % SDS. Preferably, the final nutritional composition comprises native maize starch as source of SDS.

In a further preferred embodiment, the amount of resistant starch (RS) in the final nutritional composition is preferably kept low. In this regard, aside of the carbohydrates available for digestion, both OSA starch as emulsifier and resistant maltodextrin when used as encapsulating agent, also comprise a fraction of RS as according to the method described by Englyst et al. (1992). Thus, in a preferred embodiment, the total fraction of RS in the final nutritional composition is preferably less than 30 wt %, more preferably less than 15 wt %, most preferably less than 10 wt % based on total dry weight of the final nutritional composition.

Furthermore, aside of the digestible carbohydrates available for digestion, both OSA starch as emulsifier and resistant maltodextrin when used as encapsulating agent, also comprise a fraction of rapidly digestible starch (RDS) as according to the method described by Englyst et al. (1992). Thus, in a preferred embodiment, the total fraction of RDS in the nutritional composition is preferably less than 18 wt %, more preferably less than 14 wt %, most preferably less than 12 wt % based on total dry weight of the final nutritional composition.

The final nutritional composition is preferably in compliance with food for special medical services, such as according to Food for Special Medical Purposes (FSMP) directive 1999/21/EC of 25 Mar. 1999 and as defined in Article 2.2(g) in Commission Regulation (EU) 609/2013.

In a preferred embodiment, the final nutritional composition according to the invention comprises per 100 kcal:
1-5 g lipid, more preferably 1.5-4.5 g, most preferably 2-4 g, preferably the lipid comprises sunflower oil;
optionally 2-9 g protein, more preferably 3-8 g, most preferably 4-7 g, preferably the protein comprises intact whey protein;
6-18 g digestible carbohydrates, more preferably 8-16 g, most preferably 10-14 g, preferably wherein the digestible carbohydrate fraction comprises 3.3-9.9 g slowly digestible starch (SDS), more preferably 4.4-8.8 g SDS, most preferably 5.5-7.7 g SDS;
0.3-1.1 g OSA starch, more preferably 0.4-1 g, most preferably 0.5-0.9 g;
1-2.5 g encapsulating agent, more preferably 1.4-2 g, most preferably 1.5-1.9 g, preferably wherein the encapsulating agent comprises resistant maltodextrin;
optionally 0.3-1.1 g food-grade pH adjusting agent, more preferably 0.4-1 g, most preferably 0.5-0.9 g, preferably wherein the food-grade pH adjusting agent comprises citric acid.

In a preferred embodiment, the daily dosage for a subject from 1-8 years is preferably 2-4 grams of nutritional composition (before reconstitution) per kg bodyweight. In a further preferred embodiment, the daily dosage for a subject from 8 years to end of puberty is preferably 80-200 g of the nutritional composition based on dry weight, more preferably 100-150 g, most preferably 135-150 g of the nutritional composition. In another preferred embodiment, the daily dosage for an adult subject comprises 50-250 g of the nutritional composition based on dry weight, more preferably 75-200 g, most preferably 90-150 g of the nutritional composition. In a further preferred embodiment, the composition according to the invention comprises per daily dosage 15-85 en %, more preferably 25-75 en % based on total daily energy intake, preferably wherein the total daily energy intake is 1500-3500 calories, more preferably is 1800-3000 calories.

In a preferred embodiment the final nutritional composition according to the invention comprises per daily dosage,
13-21 g lipid, more preferably 14-19 g, most preferably 15-18 g, preferably the lipid comprises sunflower oil;
optionally 21-35 g protein, more preferably 23-31 g, most preferably 25-29 g, preferably the protein comprises intact whey protein;
48-81 g digestible carbohydrates, more preferably 54-73 g, most preferably 59-68 g, preferably wherein the digestible carbohydrate fraction comprises 26-45 g slowly digestible starch (SDS), more preferably 29-41 g SDS, most preferably 32-38 g SDS;
2.7-4.5 g OSA starch, more preferably 3.0-4.1 g, most preferably 3.3-3.8 g;
6.3-10.5 g encapsulating agent, more preferably 7.0-9.5 g, most preferably 7.7-8.8 g, preferably wherein the encapsulating agent comprises resistant maltodextrin;
optionally 2.7-4.5 g food-grade pH adjusting agent, more preferably 3.0-4.1 g, most preferably 3.3-3.8 g, preferably wherein food-grade pH adjusting agent comprises citric acid.

### Applications of the Nutritional Composition

The spray-dried composition and the nutritional composition according to the invention is especially suitable for use in reducing and/or treating carbohydrate-related metabolic disorders. The present invention relates to method for reducing and/or treating carbohydrate-related metabolic disorders, comprising administering to the subject a nutritional composition according to the invention. Worded differently, the present invention relates to the use of OSA starch for the manufacture of a nutritional composition for reducing and/or treating carbohydrate-related metabolic disorders. The method or use is preferably for reducing and/or treating diabetes, glycogen storage disease (GSD), fatty acid oxidation disorders (FAOD) and/or idiopathic ketotic hypoglycemia (IKH), most preferably for use in GSD subtypes 0, III, VI and IX. These GSD subtypes are the so-called ketotic subtypes related to storage of glycogen in the liver and aside of slowly digestible carbohydrates, also require a relatively higher protein intake. In this regard, the nutritional composition is suitable for all age categories, preferably the nutritional composition is suitable for children aging 1 year and older, teenagers, adolescents, adults and elderly.

In a further preferred embodiment, the dosage regimen of the final nutritional composition is:
2-3 dosage units of the nutritional composition for subjects between 1-8 years, wherein a dosage unit is based on bodyweight and comprises per dosage unit 1-1.5 g of nutritional composition per kg bodyweight; and/or;
2-3 dosage units of the nutritional composition for subjects between 8-18 years, wherein a daytime dosage unit comprises 45-50 g of the nutritional composition based on dry weight and a nighttime dosage unit is based on bodyweight and comprises 1-1.5 g of nutritional composition per kg bodyweight;
2-3 dosage units of the nutritional composition for subjects aging 18 years or older, wherein one dosage unit comprises 45-50 g of the nutritional composition based on dry weight; and/or

In a preferred embodiment, reducing and/or treating diabetes, GSD, FAOD, and/or IKH is achieved through disease management, more preferably by reducing (the occurrence of) symptoms associated with said diseases. In a preferred embodiment, the term “reducing and/or treating” is meant herein as preventing and/or reducing (the occurrence of) symptoms and intensity of the disorders/diseases. In a further preferred embodiment, reducing and/or treating diabetes, GSD, FAOD, and/or IKH is achieved by preventing and/or reducing one or more of insomnia or sleep deprivation, impaired normoglycemia, hypoglycemia during fasting periods, hyperinsulinism, insulin resistance, gastrointestinal side effects comprising one or more of bloating, gas, diarrhoea. In a further preferred embodiment, reducing and/or treating diabetes, GSD, FAOD, and/or IKH is achieved by maintaining normoglycemia, prolonging gastric emptying rate, prolonging carbohydrate digestion, prolonging glycolytic breakdown during digestion.

The emulsified O/W composition, the spray-dried compositio and the final nutritional composition according to the invention ensure a homogenous distribution of the lipid throughout the gastric digesta. Therewith, lipid and other nutrients will be emptied from the stomach together. At arrival in the small intestine the lipid digestion products elicit a hormonal feedback response (CCK) slowing down gastric emptying of the stomach and therewith manages digestion of the food bolus and the overall glucose release.

In another embodiment, the method is a non-therapeutic method, preferably a non-therapeutic method selected from one or more of prolonging gastric emptying rate, maintaining normoglycemia, prolonging carbohydrate digestion and/or prolonging glycolytic breakdown during digestion in healthy subjects that are in need of prolonged gastric emptying or in need of prolonged carbohydrate digestion, such as sport athletes.

In a further alternative embodiment, the present invention relates to the use of a nutritional composition according to the invention in the manufacture of a product for reducing and/or treating carbohydrate-related metabolic disorders, more preferably for use in reducing and/or treating diabetes, glycogen storage disease (GSD), fatty acid oxidation disorders (FAOD), and/or idiopathic ketotic hypoglycemia (IKH), most preferably for use in liver related GSD subtype 0, III, VI and IX.

GSD patients are an especially preferred target group for administration of the final nutritional composition as GSD patients cannot (fully) break down glycogen to provide energy to the human body and thus have to consume frequent carbohydrate rich meals along with slowly digestible carbohydrate administration in order to maintain normoglycemia during the 24 hours in a day and to avoid excessive glycogen storage in the liver. This has an impact on the quality of life of the patient and family as it oftens results in sleep deprivation during the night. Current treatment options relate to supplementation of uncooked corn starch (e.g. maizena). This treatment option is suboptimal as patient still face limited fasting periods directly impacting their sleep and quality of life and may also result in extra eating moments or overfeeding. Current options are nutritionally poor, and the high carbohydrate intake leads to a disbalanced macronutrient intake and gastro-intestinal side effects, such as bloating, and other metabolic abnormalities such as lactic acidosis and disbalanced lipid profile. This is different from maintaining normoglycemia in diabetic patients whereby diabetics are still able to store and break down glycogen for energy and relates to a hormonal disorder which is usually treated by supplementing hormones while GSD cannot be treated by supplementation the deficient enzyme(s). Furthermore, diabetic patients are recommended to follow a ketogenic diet while GSD patients this would not work as these patients must have digestible carbohydrates as part of their dietary intake.

The relatively higher protein level in the final nutritional composition can be of benefit for GSD patients: with the gluconeogenesis pathway intact, protein-derived alanine can be used as an alternate source for glucose during times of fasting. Higher dietary protein intake may also improve muscle function by enhancing muscle protein synthesis and by providing a balanced ratio between carbohydrates and protein, unnecessary glycogen storage may be reduced. Furthermore, the protein in the final nutritional composition may support the prevention of hyperinsulinism, which is often observed in GSD patients due to the high carbohydrate intake. A further benefit of the relatively higher protein level in the final nutritional composition is that it improves the taste of the nutritional composition for consumption.

### EXAMPLES

The invention is illustrated in the following examples.

### Example 1. Emulsion Stability with OSA Starch as Emulsifier

In the present example, emulsification with OSA starch and protein by means of a one-step dispersion and homogenisation was compared to emulsification with OSA starch only by means of split stream process.

In the production of the first wet-phase emulsion, water-soluble components comprising protein (whey protein hydrolysate, Lacprodan DI-3091, Arla), OSA starch as emulsifier (HICAP-IMF, Ingredion) and resistant maltodextrin (hydrolysed maize starch, Nutriose FM06; Roquette) as encapsulating agent were simultaneously processed and homogenised with lipid (sunflower oil HOA; high in oleic acid) to form a wet-phase emulsion in which both OSA starch and protein are present on the emulsion interface.

In the split-stream process for preparing the second wet-phase emulsion, the homogenization/emulsification of the liquid composition comprising lipid (sunflower oil HOA; high in oleic acid), OSA starch (HICAP-IMF, Ingredion) and resistant maltodextrin (hydrolysed maize starch, Nutriose FM06; Roquette) has been performed in absence of protein. The protein (whey protein hydrolysate, Lacprodan DI-3091, Arla) was hydrated in a separate wet-phase and subsequently homogenized with the emulsified liquid composition comprising lipid, OSA starch and resistant maltodextrin by means of a split stream process to obtain the second wet-phase emulsion in which OSA starch only is present on the emulsion interface.

The stability of both wet-phase emulsions have been compared by leaving the emulsified liquid compositions to stand in a glass beaker untouched for 4 hours at room temperature. A visual comparison of the dispersion of the emulsified compositions was then carried out.

Following the processing of lipid in the presence of both protein and OSA starch, destabilization was visible within 4 hours in the first wet-phase emulsion, by the presence of a sediment layer on the bottom of the glass beaker, indicating a breakdown or ‘oiling off’ of the emulsified composition. The second wet-phase emulsion, which was subjected to a split stream process in which protein was not present during emulsification of lipid with OSA starch, remained stable for over a day and did not show sedimentation on the bottom of the glass beaker or any ‘oiling off’ compared to the first wet-phase emulsion.

In this regard, the destabilisation of the wet-phase emulsion by homogeneous processing of lipid with protein after 4 hours at room temperature, indicates that the emulsified composition will likely not remain stable when subjected to (1) a retention at 70° C. before spray drying and (2) reconstitution by the consumer, (3) consumption and (4) 3-5 hours of gastric digestion in an acid environment and thus not suitable for GSD patient to prolong gastric emptying.

### Example 2. Characteristics of Reconstituted Base Powders Comprising OSA Starch and Citric Acid, of the Reconstituted UCCS Powder and of the Reconstituted Nutritional Composition

The matrix characteristics and product stability of the reconstituted base powder (also referred to as ‘neutral base powder’ wherein the pH is 7) and base powder acidified to pH 4 with citric acid (hereinafter ‘acidic base powder’, anhydrous citric acid (Jungbunzlauer)) has been examined by visual observation, particle size distribution and microscopy over time. The base powders as referred herein do not comprise the carbohydrate fraction. A comparison to the acidified and neutral versions of the final nutritional products further comprising uncooked corn starch (maize starch, Ingredion) was also carried out.

40 grams of neutral base powder was reconstituted in 200 grams of water. The base powder was obtained by means of a split stream process in which lipid was emulsified with OSA starch after which protein in a separate wet-phase was added to the emulsified composition and subsequently spray dried. The base powder consisted of 6.5 g OSA starch/100 g of base powder, 28 g sunflower oil/100 g, 56 g whey protein hydrolysate/100 g, and 10 g resistant maltodextrin/100 g. The acidic base powder was prepared by reconstituting 40 grams of the neutral base powder together with 6.2 g of anhydrous citric acid in 200 grams of water.

The neutral nutritional composition powder consisted of 2.8 g OSA starch/100 g of the nutritional composition powder, 12 g sunflower oil/100 g, 24 g whey protein isolate/100 g, 4.4 g resistant maltodextrin/100 g, together with 56 g UCCS. 93 g of the nutritional composition powder was reconstituted in 200 g water. The reconstituted acidic nutritional composition powder consisted of 2.5 g OSA starch/100 g of the nutritional composition powder, 11.5 g sunflower oil/100 g, 23 g whey protein isolate/100 g, and 4 g resistant maltodextrin/100 g, and 53 g UCCS (maize starch, Ingredion). In addition, a sample was prepared comprising 56 grams UCCS which was reconstituted in 215 grams of water.

### Visual Observation

The reconstituted acidic and neutral base powders as well as acidic and neutral composition powder and reconstituted UCCS powder were visually observed directly after and 4 hours following reconstitution in water.

The UCCS sample showed sedimentation of the insoluble starch granules almost directly after reconstitution, forming a viscous and sticky sediment. No foaming was observed. During reconstitution of all other powders a foam formed that did not easily disintegrate. Foaming was more distinct on the reconstituted acidic base powder and reconstituted acidic nutritional composition powder. In this regard, the acidic powders appeared more viscous upon reconstitution. The foaming was not unexpected as a higher viscosity increases foam stability due to a greater resistance to drainage and consequently could explain more distinct foaming seen for the acidic powders upon reconstitution.

The neutral base powder showed only little sedimentation after 4 hours following reconstitution and no oiling-off of lipid was observed in the base powder. The reconstituted neutral nutritional composition powder also showed a starch sediment on the bottom of the glass beaker, although this sediment layer was less visible compared to the UCCS sample. For both the reconstituted acidic base powder and the reconstituted acidic nutritional composition powder a water layer was formed, respectively on the top and bottom of the solution, within 2 hours after reconstitution. When the reconstituted acidic powders were stirred with a spoon, the products returned to its original state having a homogenous dispersion without any water layers.

### Microscopy Imaging

Furthermore, the reconstituted powders were observed under the microscope after 4 hours following reconstitution. Eight samples were prepared and observed:
(1) undiluted neutral base powder at 10× magnification,
(2) 10× diluted neutral base powder at 10× and 40× magnification,
(3) undiluted acidic base powder at 10× magnification,
(4) 10× diluted acidic base powder at 10× and 40× magnification,
(5) undiluted neutral nutritional composition powder at 10× magnification,
(6) 10× diluted neutral nutritional composition powder at 10× and 40× magnification,
(7) undiluted acidic nutritional composition powder at 10× magnification, and
(8) 10× diluted reconstituted acidic final nutritional powder at 10× and 40× magnification.

No microscopic images were obtained from the UCCS sample due to uneven distribution of the sample and phase separation of the UCCS sample.

As expected, the microscopic images of the neutral base powder showed the presence of emulsion droplets of a consistent size without signs of destabilization, i.e. agglomeration or coalescence. The protein added to emulsified liquid composition was hydrated in a split stream process upon which is it was added to the base powder after emulsification and thus could not be observed on the microscopic images.

Furthermore, the microscopic images of the reconstituted neutral nutritional composition powder showed the presence of larger starch granules entrapped between the emulsion droplets of consistent size and thereby showing a homogenous dispersion of lipid droplets coated with OSA starch and the UCCS granules as main carbohydrate source. The micrographs of the neutral nutritional composition powder showed fewer starch granules in a well dispersed emulsion matrix.

The micrograph of the undiluted acidic base powder shows the formation of a gel network, that upon 10× dilution seems to fall apart into clusters of particles. A similar network can be seen for the acidic nutritional composition powder. In the micrograph of the acidic nutritional composition powder a large number of starch granules can be seen, which appear to be entrapped in the gel network.

It is hypothesized that a gel network can only hold a certain amount of water, expelling residual water by syneresis, explaining formation of the water layer in the acidic matrices. The entrapment of a high quantity of starch granules, that tend to sediment, in the acidic nutritional composition powder could cause the gel network to form in the bottom phase. Entrapment of the starch granules in the gel network could also explain the absence of a starch sediment for the acidic nutritional composition powder. Thereby, entrapment of the starch in the gel network ensures a more homogeneous distribution of starch granules throughout the product. Consequently, all nutrients in the nutritional composition powder can be consumed simultaneously and formation of a thick carbohydrate slurry is prevented.

### Particle Size Distribution

Finally, the particle size distribution of the neutral base powder and acidic base powders were quantified directly after and 4 hours following reconstitution in water. The droplet size distribution of the base powder samples were determined using a laser-light diffraction unit (Mastersizer 2000, Malvern Instruments Ltd, Worcestershire, UK) for example by the method described in Michalski et al., 2001, Lait, 81, 787-789. The size distribution was obtained using polydisperse analysis.

A particle size between 0.2 and 5 μm was observed in the neutral base powder thereby verifying the visual and microscopic observations that the neutral base powder remained stable and homogenously dispersed without agglomeration or sedimentation after 4 hours.

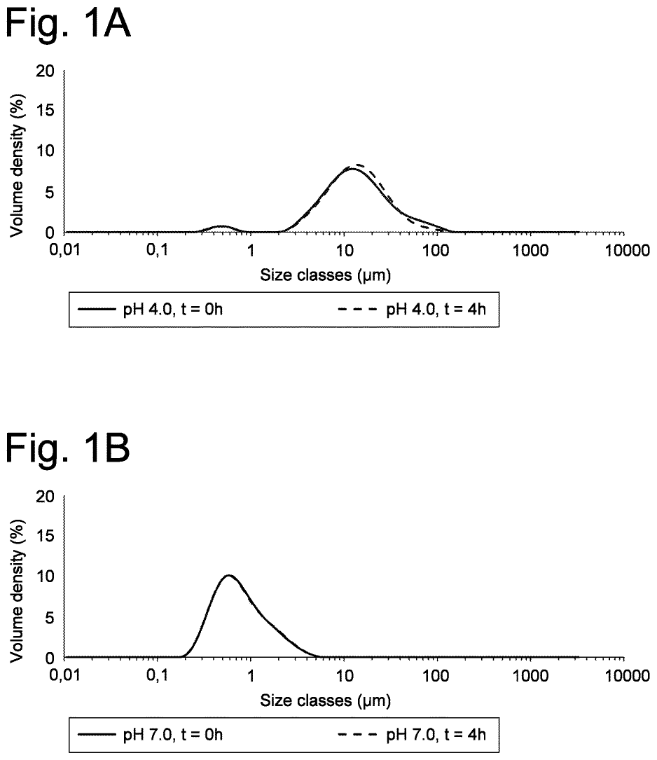

The acidic base powder at pH 4.0 shows increased particle size matching the size class of aggregates observed by microscopic analysis of the acidic base powder at a 10× dilution. The particle size distribution remains stable over time (4 hours) for both the reconstituted base powder at neutral and acidic pH (see FIG. 1A en FIG. 1B).

### Example 3. OSA Starch Emulsified Oil-In-Water Emulsion is Better Resistant to the Acidic Gastric Environment than a Protein Emulsified Oil-In-Water Emulsion

An in vitro gastric digestion simulation was performed, and the stability of an OSA-starch emulsified liquid composition was compared to a whey protein isolate-emulsified composition before and during in vitro gastric digestion. The primary aim was to predict emulsion stability in the gastric environment.

### Preparation of the OSA Starch Based Oil-In-Water Emulsion

OSA-starch (225 g; emulsifier) was dry mixed with soluble corn fibre (Promitor 70; also referred to as resistant maltodextrin) (468 g; encapsulating agent) and dissolved in water (1200 g) at 80° C. using the ViscoJet® agitator at low shear rate to avoid foam formation to obtain a water phase. Sunflower oil (1,080 g) was separately heated to 55° C. and slowly added to the water phase by using a rotor-stator system (Ultra-Turrax®, IKA T50, Germany) at 10,000 rpm for 5 minutes to obtain an oil-in-water pre-emulsion. Finally, the pre-emulsion was processed by the high-pressure homogenizer GEA®-Pony with two-step homogenization at 150/50 bar single-pass and feed flow of 80 litre/hr. To obtain the final O/W emulsion in a dry powder, the emulsion was spray dried using a GEA® Mobile Minor spray dryer with 1-7 kg/hour water evaporative capacity equipped with two-fluid nozzle type (Module System Range 970, Schlick®) for atomization wherein the inlet air temperature was set at 190° C. and outlet air temperature was set at 90-94° C.

The lipid content was fixed at 60 wt %, OSA-starch content at 14 wt % and resistant maltodextrin at 26 wt % based on total dry matter content wherein an OSA-starch to lipid ratio of 1:4.3 in dry matter was maintained. The OSA-starch base powder was reconstituted in demineralized water at a ratio of 1:15 and ready to use in the semi-dynamic gastric digestion model.

### Preparation of Whey Protein Isolate (WPI) Based Oil-In-Water Emulsion

Oil-in-water emulsion stabilised by WPI was used as a control in this study. The WPI-O/W emulsion was obtained following the method described by Mantovani et al. (2013). In summary, WPI (Lacprodan DI-9212; 105 g) was dissolved in demineralized water (3395 g) at room temperature with an overhead stirrer (IKA EUROSTAR 20 digital, Germany) for 2 hours to obtain a water phase. Sunflower oil (1500 g) was added slowly to the water phase to create a pre-emulsion using a rotor-stator system (Ultra-Turrax®, IKA T50, Germany) at 10,000 rpm for 5 minutes. Finally, the pre-emulsion was processed by the high-pressure homogenizer GEA®-Pony with two-step homogenization at 400/50 bar, single pass and feed flow of 80 litre/hr. The oil phase content was fixed at 30 wt % and WPI at 2.1 wt %.

### Semi-Dynamic Gastric Digestion Model

The simulation of the gastric digestion phase was done using a semi-dynamic digestion model as according to the standardised in vitro digestion method described in Mulet-Cabero et al. (2020). Gastric conditions included an increasing acidic pH for 120 minutes in the presence of gastric enzymes. Simulated salivary fluid (SSF) and simulated gastric fluid (SGF) were prepared according to the protocol described in Brodkorb et al. (2019); Mulet-Cabero et al. (2020). SSF contained salivary a-amylase at a concentration of 150 U/ml and SGF contained porcine pepsin at a concentration of 4000 U/ml and bacterial lipase at a concentration of 120 U/ml.

A sample of the OSA starch O/W emulsion (20 g) was placed into a plastic cup that was kept in a water bath (Fisherbrand™ Isotemp™), thermoregulated at 37° C. and the OSA-starch sample was mixed with an oral mixture consisting of SSF using an overhead stirrer (IKA EUROSTAR 20 digital, Germany) at 30 rpm for 10 seconds. The volume of the added SSF corresponded to the total solid content of emulsion sample as according to the method described in Mulet-Cabero et al. (2020).

The mixture was then subjected to simulated gastric digestion. Two solutions were added at a constant rate with separate feeding pumps (Flocare® Infinity™ pump):
(1) 18 ml of electrolyte simulated gastric fluid (eSGF) solution was added at 9 ml/h rate. The gastric mixture at pH 7 contained 17.55% demineralized water, 2.40% 1 M HCl and 0.06% 0.3 M CaCl2(H2O)2. The amount of HCl added was based on the amount of acid needed to reach pH 2 at the end of the gastric digestion.
(2) 2 ml SGF enzyme solution containing pepsin and lipase was added at 1 ml/h rate.

The simulation of the gastric emptying (GE) was based on caloric density. Normally, a linear GE rate of 2 kcal/min/500 ml, which is considered the average caloric content that is emptied in vivo in a regulated manner for an average food volume of 500 mL, is used and scaled down for the reduced volume system (Mulet-Cabero et al., 2020). For the matter of simplicity and to be able to compare the outcome of the different emulsion samples subjected to this semi-dynamic digestion model, the in vitro gastric digestion process was always fixed at 120 minutes. As a consequence, the gastric emptying rate changed from 2 kcal/min to 1.79 kcal/min for the digestion of OSA-starch O/W emulsion sample. This modification made it possible to have a similar profile in terms of the amount of both SGF and enzyme solutions being delivered, together with the amount of sample that was emptied during simulated gastric digestion. Gastric emptying (GE) was simulated by taking a sample every 24 min, further referred to as GE1—24 min, GE2—48 min etc. up until 120 minutes of digestion. Samples were collected from the bottom of the plastic cup using a manual single channel pipette with a tip internal diameter of 2 mm approximating the upper limit of particle size that has been seen to pass through the pyloric opening into the duodenum (Mulet-Cabero et al., 2019). An aliquot of these GE samples was used for microscopic and particle size analysis.

For the simulated digestion of control WPI O/W emulsion the same set-up was used as described above. The only modification was made in the starting amount of emulsion sample to be subjected to in vitro gastric digestion in order to have the same flow rate profile of both eSGF and enzyme solutions as for OSA-starch emulsion sample.

### Particle Size Distribution

The droplet size distribution and average lipid droplet size of the initial samples before digestion and the samples that subjected to digestion were determined using a laser-light diffraction unit (Mastersizer, Malvern Instruments Ltd, Worcestershire, UK). The size distribution was obtained using polydisperse analysis while droplet size measurements were recorded as average mean diameter D50 and volume mean diameter (D_{4,3}) to observe the effect of gastric digestion on the change of the oil droplet size distribution. Every sample was quantified in duplicate.

### Light Microscopy

A light microscope (Zeiss Axioskop 2) was used to observe structural changes in the emulsion samples before and during digestion and to verify the obtained results from the particle size distribution analysis. An aliquot of each sample (3 μL) was placed on a microscope slide and covered by a cover slip. Dilutions were performed at a ratio of 1:25 with demineralized water for reconstituted powders (1:10 ratio). The images were captured with Axovision LE64 software at 100× times magnification.

### Results

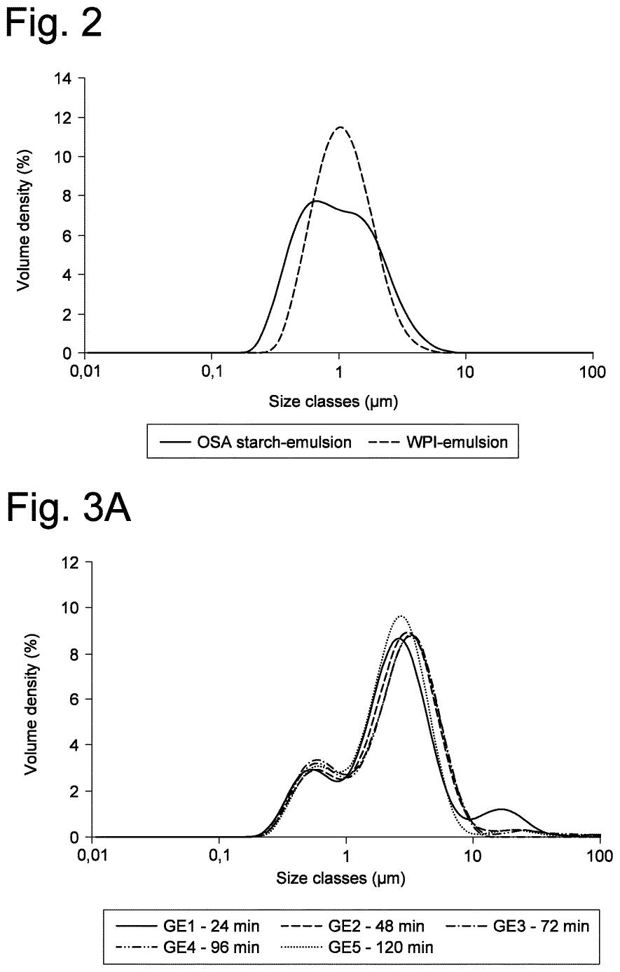

FIG. 2 shows that particle size distribution of both emulsions after reconstitution and before gastric digestion. As can be seen the particle size distribution for both emulsions are similar but the particle size distribution of the OSA starch O/W emulsion shows a larger spread in particle size ranging from 0.15-9.2 μm while the particle size for the WPI-O/W emulsion ranges from 0.2-7 μm. Reconstitution of both emulsions thus did not result in any agglomerations and remained dispersed in the samples.

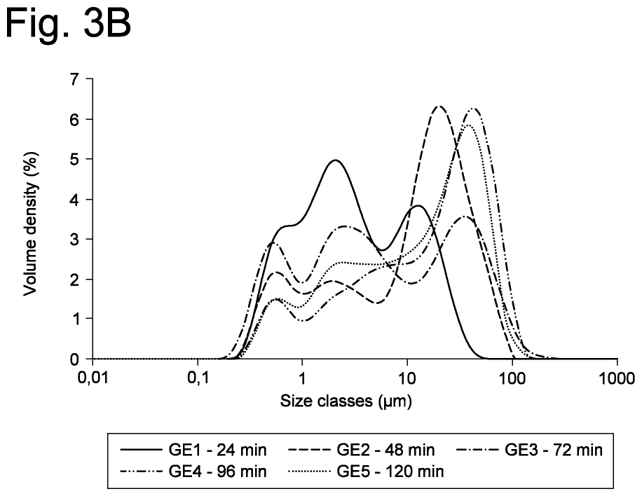

FIGS. 3A and 3B show the comparison between an emulsion stabilised by OSA starch and an emulsion stabilised by WPI during simulated gastric digestion. As can clearly be observed, the particle size distribution for the OSA-starch emulsion (FIG. 3A) is much less spread with the majority of particles having a size between ±0.4 μm and ±9 μm and shows a large overlap in size distribution compared to the emulsion before being subject to gastric digestion simulation (see FIG. 2). In contrast, FIG. 3B shows a large variation is the particle size distribution of the WPI-O/W emulsion and the particle size distribution ranges between 0.5 μm and even up to 100 μm while the initial particle size before being subjected to simulated gastric digestion had a particle size ranging from 0.2-7 μm. Hence, the data suggests agglomeration of lipid droplets in the WPI-O/W emulsion under gastric digestion conditions.

Referring to FIG. 3A, it should be noted that the GE1 sample of the OSA-starch O/W emulsion showed a second peak at around 20-30 μm, which could represent the appearance of flocculated droplets during the initial stage of simulated gastric digestion. The microscopy analysis of the GE1 sample, however, did not confirm this as the presence of flocculated or coalesced droplets with the size of approximately 20 μm was not observed.

Looking in further detail, the averaged D50 based on the measurements taken from samples GE1-GE5 was 2.5 μm for the OSA starch-O/W emulsion during simulated gastric digestion and the D50 of each sample GE1 to GE5 differed at most 8% from the averaged D50. Concerning the WPI-O/W emulsion, the averaged D50 based on samples GE1-GE5 was 10.4 μm during simulated gastric digestion and the D50 of each sample GE1 to GE5 differed at least 41% from the averaged D50.

In addition, the average volume mean diameter D[4,3] based on the measurements taken from the 5 samples (GE1-GE5) during simulated gastric digestion was 3.24 μm for the OSA starch-O/W emulsion. The volume mean diameter D[4,3] of each sample GE1 to GE5 differed at most 14% from the average volume mean diameter. Concerning the WPI-O/W emulsion, the average volume mean diameter D[4,3] based on samples GE1-GE5 was 18.2 μm and the D[4,3] of each sample GE1 to GE5 differed up to 66% from the average volume mean diameter showing a large variation in the particle size distribution.

In addition to quantification of the particle sizes, microscopic images were taken of the emulsion samples during gastric digestion simulation at 24 minutes, 72 minutes and after 120 minutes. The main observation was the confirmation of agglomeration of particles in the WPI-O/W emulsion which were already visible at 24 minutes during gastric digestion simulation. A substantial amount of free oil phase could be observed within the first hour of simulated gastric digestion, displaying the instability of WPI-O/W emulsion when subjected to gastric enzymes and salts. Flocculation of oil droplets together with the release of free oil could plausibly be attributed to the proteolysis of the protein-coated interfacial layer by pepsin and salts present in the simulated gastric fluid.

Yet, the OSA starch-O/W emulsion remained stable without any noticeable instability or oiling-off throughout simulated gastric digestion. Particles in the OSA starch-O/W emulsion were mostly of the same size and only a small variation in particle size was observed after 120 minutes compared to the many large agglomerations present in the WPI-O/W emulsion after 120 minutes of digestion.

Taken together, these results indicate that the OSA starch-O/W emulsion is less susceptible to lipolysis under gastric conditions and thereby can better withstand the gastric environment than a WPI stabilizid O/W emulsion. These results indicate that an OSA starch stabilized emulsion can remain stable in an acid environment and remain more homogenously spread in a nutritional composition thereby able to elicit the CCK mechanism and delaying the gastric emptying already early after intake of the nutritional composition and oral ingestion.

### Example 4. OSA Starch Base Powder is Better Resistant to the Acidic Gastric Environment than WPI Base Powder

An in vitro gastric digestion simulation was performed similar to Example 3 and the stability of reconstituted OSA-starch base powder was compared to a reconstituted whey protein isolate base powder before and during in vitro gastric digestion.

OSA—Starch Based Oil-In-Water Emulsion=Basepowder A

First, 1740 g OSA starch (HICAP-IMF, Ingredion) was dry-blended with 2772 g resistant maltodextrin (Nutriose FM06, Roquette) and subsequently dissolved in 8030 g water at 40° C. using an overhead stirrer by slowly adding the dry blend to the water to prevent foaming and prevent creating a vortex. Then, pH of the water phase was adapted to 4.2 using 10% citric acid solution. 7458 g sunflower oil was heated to 55° C. and added to the water phase using the overhead stirrer and the subsequent emulsion was homogenized using two-stage homogenization at 150:50 bar and a temperature of 70° C. Then, the emulsion was thermally processed using direct steam injection with a pre-heat temperature of 70° C., main heat temperature of 126° C. (2-2.6 seconds hold time) and cooled to 65° C. The processed liquid was subsequently spray-dried using a GEA® Mobile Minor spray dryer to obtain the base powder, further referenced as Basepowder A in this example.

### WPI Based Oil-In-Water Emulsion=Basepowder B

First, 1175 g whey protein isolate (WPI; Lacprodan is dissolved in 9868 g water at 40° C. using an overhead stirrer by slowly adding the WPI to the water to prevent foaming and prevent creating a vortex. 2427 g resistant maltodextrin (Nutriose FM06, Roquette) was subsequently dissolved in the protein solution. Then, pH of the water phase was adapted to 4.2 using 10% citric acid solution. 6529 g sunflower oil was heated to 55° C. and added to the water phase using the overhead stirrer and the subsequent emulsion was homogenized using two-stage homogenization at 150:50 bar and a temperature of 70° C. Then, the emulsion was thermally processed using direct steam injection with a pre-heat temperature of 70° C., main heat temperature of 115° C. (5.6 seconds hold time) and cooled to 65° C. The processed liquid was subsequently spray-dried using a GEA® Mobile Minor spray dryer to obtain the base powder, further referenced as Base powder B in this example.

### Sample Preparation for Digestion Experiment

Following the instructed method of use for the final product, that indicated to add 92 g in a measurement beaker and add water until 260 ml total, which equals to a dry weight content of 35%. During the experiment, 35 g of each base powder was reconstituted in 65 g of water and stirred using a magnetic stirrer until a homogenous solution was obtained. This was done in the same manner for base powder A and B.

### Semi-Dynamic Digestion

50 g of each reconstituted base powder solution was placed in a glass beaker inside a water bath, maintaining an internal temperature of 37° C. (water bath was set at 39° C.). To begin salivary digestion, the base powder solution was mixed with the electrolyte simulated salivary fluid (eSSF) described by Mulet-Cabrero et al., 2020, pre-warmed to 37° C. The volume of the added eSSF corresponded to the total solid content of 35% (w/w) of the base powder solution. This mixture was stirred using an overhead stirrer at 30-50 rpm for 5 minutes and samples were taken for particle size distribution analysis.

The remaining base powder solution was then subjected to simulated gastric digestion. Two solutions were added at a constant rate with separate feeding pumps -: (1) 57 ml of electrolyte simulated gastric fluid (eSGF) solution was added at 36 ml/h rate. The electrolyte simulated gastric fluid (eSGF) included KCl, KH2PO4, NaCl, NaHCO₃, MgCl2(H2O)6 and (NH4)CO3, and was prepared by mixing the different electrolyte stock solutions at concentrations described in Mulet-Cabrero et al, 2020. The amount of HCl added corresponded to the amount of acid needed for the product to reach pH 2 at the end of the gastric digestion. (2) 6.2 ml of gastric enzyme solution containing pepsin and lipase were added at 4 ml/h rate.

Gastric emptying (GE) was simulated by collecting a sample every 24 min, further referred to as GE1—24 min, GE2—48 min etc. up until 120 minutes of digestion. Samples of 12 ml were collected from the bottom of the glass beaker using a manual single channel pipette. Aliquots of these samples at each time point were used to determine particle size distribution.

### Results

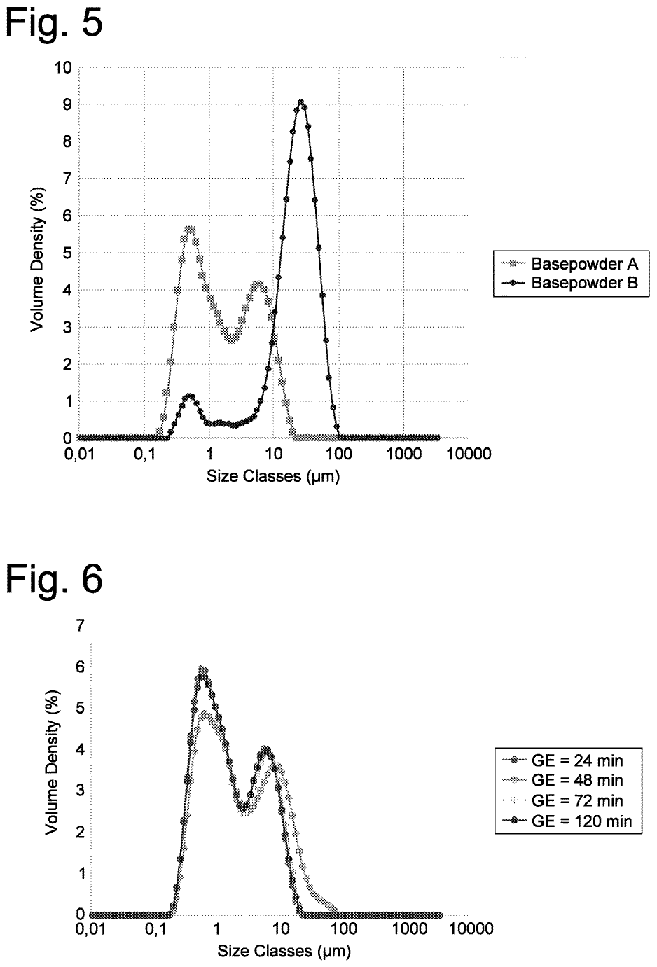

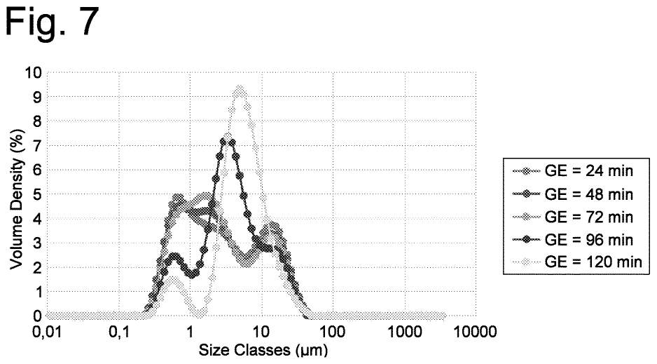

Base powder A remained stable in solution (at room temperature 25° C.), no phase separation was observed between the time of solubilization and the beginning of the digestion experiment. Before being subjected to the digestion experiment the reconstituted sample of base powder B remained stable in solution, no phase separation was observed, see FIG. 5. During digestion, the particle size distribution of base powder A solution remained the same and this stability can also be observed in FIG. 6. In comparison, the average particle size of base powder B solution changed to a narrower distribution of larger particles during gastric digestion (FIG. 7). The destabilization of the base powder B solution during the gastric digestion resulted in an extensive phase separation. In conclusion, OSA-starch was an effective stabiliser for the emulsion during digestion. The emulsified oil stabilised by the OSA starch remained homogeneously distributed throughout the gastric system for the whole duration of the digestion experiment (120 min).

### Example 5. Method Preparing the Nutritional Compositions

First, 3779 g OSA starch (HICAP-IMF, Ingredion) was dry-blended with 6020 g resistant maltodextrin (Nutriose FM06, Roquette) and subsequently dissolved in 18000 g water at 40° C. using an overhead stirrer by slowly adding the dry-blend to the water to prevent foaming and prevent creating a vortex. Then, pH of the water phase was adapted to 4.2 using 10% citric acid solution.

16199 g sunflower oil was heated to 55° C. and added to the water phase using the overhead stirrer and the subsequent emulsion was homogenized using two-stage homogenization at 150:50 bar and a temperature of 70° C.

32455 g whey protein hydrolysate (Arla DI-3091) was dissolved in 64911 g water at 40° C. using an overhead stirrer by slowly adding the protein to the water to prevent foaming and prevent creating a vortex.

Then, the emulsion and the protein solution were combined in one tank under agitation. The mixture was then thermally processed using direct steam injection with a pre-heat temperature of 70° C., main heat temperature of 126° C. (2-2.6 seconds hold time) and cooled to 70° C.

The processed liquid was subsequently spray-dried using a high pressure nozzle to obtain the base powder.

Finally, 3366 g native maize starch (Ingredion) was added to 2600 g base powder by first adding half of the maize starch and half of the base powder, then 30 g of orange flavour and 3 g of sucralose were added, followed by adding the second half of the maize starch and the base powder. The mixture was dry-blended for 25 minutes to ensure homogeneity and sieved with a 2 mm sieve. The final product was then ready for packaging.

The same method of manufacture as described here above was applied to obtain the base powder for the acidic nutritional composition with a pH 4. Then 3152 g of native maize starch (Ingredion) was added to 2440 g base powder by first adding half of the maize starch and half of the base powder, then 376 g citric acid, 28.20 g orange flavour and 2.82 g sucralose were added, followed by adding the second half of the maize starch and the base powder. The mixture was dry-blended for 25 minutes to ensure homogeneity and sieved with a 2 mm sieve. The final product was then ready for packaging.

### Example 6. Proof-of-Concept Study on the Nutritional Composition Comprising OSA Starch in Healthy Subjects

### Study Protocol

A randomised, controlled, blind, cross-over, single-centre proof-of-concept study was conducted comparing the glucose response as well as indirect gastric emptying rate of the study products (neutral prototype and acidic prototype) compared to uncooked corn starch alone in 15 healthy adult subjects. Subjects were screened upon signing informed consent and screening was based on a list with in- and exclusion criteria. Several examples of exclusion criteria included: abnormal blood glucose levels at screening, known history of gastrointestinal diseases and food allergies, extreme dietary habits such as ketogenic diet or Atkins diet. Subjects eligible for participation were randomly allocated to receive one study product per visit. In total, each subject received three study products across three different study visits following a randomisation scheme and between each visit there was a break (washout) of at least 48 hours.

On each study visit, a fasted baseline blood sample was collected prior to study product consumption. Study products were matched on available carbohydrate content as well as total volume and were mixed with paracetamol before consumption by the study subjects. After consumption, blood samples were collected at 30-minute intervals for 6 hours post-prandial. According to a standardized schedule, subjects were given a glass of 150 mL water at interval t=180 minutes, t=240 minutes and t=300 minutes (mandatory). Blood samples were analysed for glucose and paracetamol levels. Paracetamol levels were analysed as an indirect measure of gastric emptying as paracetamol is directly taken up into the bloodstream from the stomach. 15 subjects in total completed the proof-of-concept study per protocol.

### Study Products

1. Uncooked Corn Starch (UCCS) comprising per 100 g: 89 g carbohydrates, 0.45 g protein, 0.13 g lipid. The sample was then prepared by dry-blending 56 g of UCCS powder with 0.5 g orange flavour and subsequently reconstituted in 215 g water.
2. Neutral prototype (pH 7) comprising per 100 g: 56.1 g carbohydrates (UCCS), 24.1 g protein (whey protein hydrolysate (from cow's milk)), 12.1 g lipid (sunflower oil), 4.5 g resistant maltodextrin, 2.8 g OSA starch, 0.5 g orange flavour and 0.05 g sweetener (sucralose). The sample was prepared according to the method as explained in Example 4. 93 g of the neutral prototype was reconstituted in 200 g water.
3. Acidic prototype (pH 4) comprising per 100 g: 52.5 g carbohydrates (UCCS), 6.3 g organic acids (citric acid), 22.5 g protein (whey protein hydrolysate (from cow's milk)), 11.3 g lipid (sunflower oil), 4.2 g resistant maltodextrin, 2.6 g OSA starch, 0.5 g orange flavour and 0.05 g sweetener (sucralose). The acidic prototype was prepared as explained in Example 4. 99 g of the acidic prototype was reconstituted in 200 g water.

Study products were matched on available carbohydrate content (50 g) and total volume upon preparation (260 mL).

Study products were matched on available carbohydrate content (50 g) and total volume upon preparation (260 mL).

### Statistical Analysis

Glucose profiles of the three treatment groups (UCCS, pH 7 and pH 4) were compared using longitudinal analysis using a linear mixed-effects model and taking into account the subject variability across timepoints, treatment groups and visits.

### Results

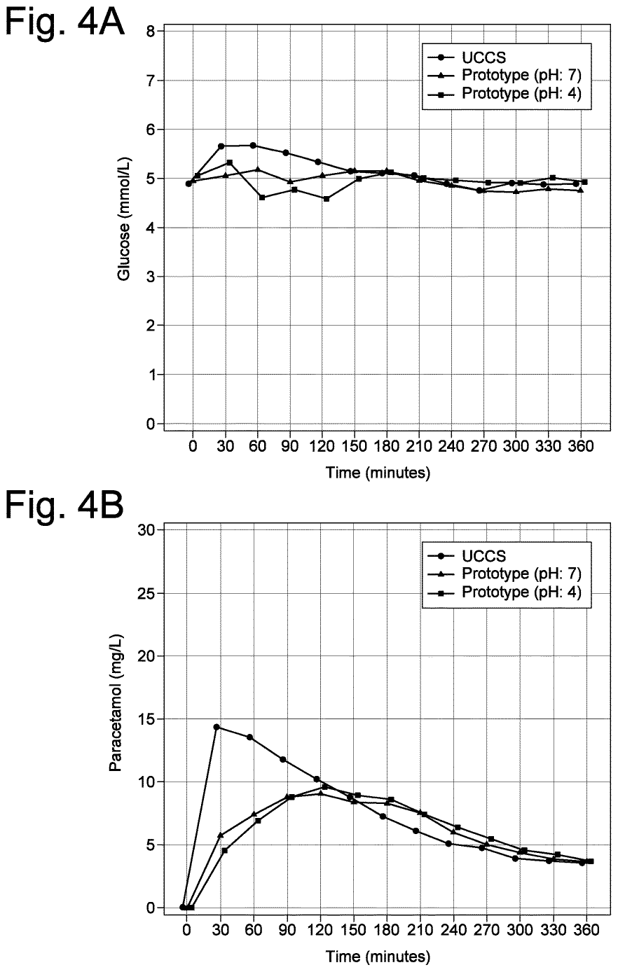

FIG. 4A en 4B show the blood glucose levels over time of glucose and paracetamol respectively. Glucose levels of both the acidic and neutral prototype show stable blood glucose values after consumption while UCCS showed an increase in blood glucose level after consumption (FIG. 4A).

Blood glucose profile of both prototypes were significantly lower than the glucose profile of UCCS, see Table 1.

TABLE 1
Statistical analysis of GSD prototypes versus
UCCS of glucose blood levels over time.

| Comparison | Estimate | CI* | p-value |
|---|---|---|---|
| UCCS vs. neutral prototype (pH 7) | 0.28 | 0.03:0.53 | 0.026 |
| UCCS vs acidic prototype (pH 4) | 0.40 | 0.15:0.66 | 0.002 |

In addition, blood paracetamol levels, as an indirect measure for gastric emptying, were significantly lower compared to the paracetamol levels of the UCCS group, thereby showing the controlled and slower release of nutrients from the stomach, see FIG. 4B and Table 2.

TABLE 2
Statistical analysis of GSD prototypes versus
UCCS of paracetamol blood levels over time

| Comparison | Estimate | CI* | p-value |
|---|---|---|---|
| UCCS vs. neutral prototype (pH 7) | 1.12 | 0.55:1.68 | <0.001 |
| UCCS vs acidic prototype (pH 4) | 1.17 | 0.60:1.73 | <0.001 |

### Conclusion

Overall glucose profiles of both prototypes were significantly lower than the glucose profile of UCCS. Furthermore, the paracetamol peak for the prototypes was lower and had a longer duration after ingestion of the study products indicating that gastric emptying is better controlled by both prototypes compared to UCCS alone.

### Example 7. Formulations of a Neutral (pH 7) and an Acidified (pH 4) Nutritional Composition

An exemplary neutral nutritional composition was formulated comprising per 100 grans 414 kcal and:
53 g uncooked corn starch (UCCS)
20 g whey protein hydrolysate (from cow's milk)
12 g sunflower oil
2.5 g OSA starch
4 g resistant maltodextrin

An exemplary acidic nutritional composition was formulated comprising per 100 grams 406 kcal and:
53 g uncooked corn starch (UCCS)
23 g whey protein hydrolysate (from cow's milk)
11 g sunflower oil (high in oleic acid)
2.5 g OSA starch
4 g resistant maltodextrin
6.3 g citric acid

1.-15. (canceled)

16. A method for preparing an emulsified oil-in-water (O/W) composition comprising emulsifying lipid with a first aqueous phase and octenyl succinic anhydride substituted starch (OSA) starch to obtain an emulsified O/W composition, wherein the dry weight ratio of OSA starch to lipid is in the range from 1:3 to 1:5 and wherein less than 4 wt % protein is present, based on total dry weight of the emulsified O/W composition and wherein the emulsified O/W composition comprises less than 10 wt % digestible carbohydrates based on total dry weight of the emulsified O/W composition, wherein the method further comprises:
adding an encapsulating agent to the emulsified O/W composition;
optionally adding a second aqueous phase comprising protein to the emulsified O/W composition and optionally pasteurizing the emulsified O/W composition;
spray-drying the emulsified O/W composition, to obtain a spray-dried composition;
wherein the spray-dried composition comprises lipid-containing particles which have a lipid core and wherein said lipid core is coated with OSA starch, and wherein the lipid core and OSA starch coating comprise less than 4 wt % protein altogether, based on total dry weight of the spray-dried composition.

17. The method according to claim 16, wherein the dry weight ratio of OSA starch to lipid is in the range from 1:3.5 to 1:4.5.

18. The method according to claim 16, wherein emulsification of lipid with the first aqueous phase and OSA starch is performed at a pH in the range of 3-6.5.

19. The method according to claim 16, wherein the encapsulating agent is added to the emulsified O/W composition and/or to the second aqueous phase.

20. The method according to claim 16, wherein the encapsulating agent has a glycaemic index in the range of 0 to 50.

21. The method according to claim 16, wherein less than 4 wt % digestible carbohydrates based on total dry weight is present in the emulsified O/W composition prior to being subjected to spray-drying.

22. A nutritional spray-dried composition obtainable by the method according to claim 16, comprising lipid, OSA starch, an encapsulating agent and optionally protein, wherein the composition comprises less than 10 wt % digestible carbohydrates based on total dry weight of the composition.

23. The nutritional spray-dried composition according to claim 22, wherein the encapsulating agent has a glycaemic index in the range of 0 to 50.

24. The nutritional spray-dried composition according to claim 22, wherein the composition comprises less than 10 wt % lipid-containing particles of which the core is not coated with OSA starch based on total lipid weight in the nutritional composition.

25. The nutritional spray-dried composition according to claim 22, wherein the composition comprises based on total dry weight of the composition:
10-55 wt % lipid;
2-20 wt % OSA starch;
2-24 wt % encapsulating agent; and
20-70 wt % protein.

26. A method for reducing and/or treating diabetes, glycogen storage disease (GSD), fatty acid oxidation disorder (FAOD) and/or idiopathic ketotic hypoglycemia (IKH) in a subject in need thereof wherein the method comprises administering the nutritional spray dried composition to the subject.

27. The method according to claim 26, wherein the method for reducing and/or treating diabetes, GSD, FAOD, and/or IKH comprises preventing and/or reducing one or more of insomnia or sleep deprivation, impaired normoglycemia, hypoglycemia during fasting periods, hyperinsulinism, insulin resistance, gastrointestinal side effects comprising one or more of bloating, gas, diarrhoea and wherein reducing and/or treating diabetes, GSD, FAOD, and/or IKH further comprises one or more of maintaining normoglycemia, prolonging gastric emptying rate, prolonging carbohydrate digestion, prolonging glycolytic breakdown during digestion in a subject in need thereof wherein the method comprises administering the nutritional spray dried composition to the subject.
